(19) United States
(12) Patent Application Publication
(10) Pub. No.: US 2009/0306586 A1
(43) Pub. Date: Dec. 10, 2009

(54) LATERALLY-EXPANDABLE ACCESS CANNULA FOR ACCESSING THE INTERIOR OF A HIP JOINT

(76) Inventors: Lynette Ross, Mountain View, CA (US); Dwayne Dupree, Oakland, CA (US); James Flom, San Carlos, CA (US); Roger Pisarnwongs, Valencia, CA (US)

Correspondence Address: Mark J. Pandiscio;Pandiscio & Pandiscio, P.C., 470 Totten Pond Road, Waltham, MA, 02451, US

(21) Appl. No.: 12/395,137

(22) Filed: Feb. 27, 2009

Related U.S. Application Data
(60) Provisional application No. 61/067,663, filed on Feb. 29, 2008.
(60) Provisional application No. 61/135,471, filed on Jul. 21, 2008.

(51) Int. Cl. A61M 31/00; A61F 11/00

(52) U.S. Cl. 604/93.01; 606/108

(57) ABSTRACT

A laterally-expandable access cannula comprising:
an elongated body have a distal end, a proximal end and a lumen extending between the distal end and the proximal end, the distal end of the access cannula comprising a plurality of fingers tapering inwardly as they extend distally, and the elongated body having an internal thread extending along at least a portion of the length of the lumen; and
an inner sleeve disposed within the lumen of the elongated body, the inner sleeve having an external thread extending along at least a portion of its length, the external thread of the inner sleeve being in engagement with the internal thread of the elongated body, such that rotation of the inner sleeve causes the inner sleeve to move distally relative to the elongated body, whereby to cam open the plurality of fingers of the elongated body.

### REFERENCE TO PENDING PRIOR PATENT APPLICATIONS

This patent application claims benefit of:

(i) pending prior U.S. Provisional Patent Application Ser. No. 61/067,663, filed Feb. 29, 2008 by Lynette Ross et al. for ATRAUMATIC ACCESS CANNULA FOR ACCESSING THE INTERIOR OF A HIP JOINT (Attorney's Docket No. FIAN-21 PROV); and

(ii) pending prior U.S. Provisional Patent Application Ser. No. 61/135,471, filed Jul. 21, 2008 by Lynette Ross et al. for LATERALLY-EXPANDABLE ACCESS CANNULA FOR ACCESSING THE INTERIOR OF A HIP JOINT (Attorney's Docket No. FIAN-23 PROV).

The two above-identified patent applications are hereby incorporated herein by reference.

### FIELD OF THE INVENTION

This invention relates to surgical methods and apparatus in general, and more particularly to surgical methods and apparatus for treating a hip joint.

### BACKGROUND OF THE INVENTION

### The Hip Joint in General

The hip joint is a ball-and-socket joint which movably connects the leg to the torso. The hip joint is capable of a wide range of different motions, e.g., flexion and extension, abduction and adduction, medial and lateral rotation, etc. See FIGS. 1A, 1B, 1C and 1D.

With the possible exception of the shoulder joint, the hip joint is perhaps the most mobile joint in the body. Significantly, and unlike the shoulder joint, the hip joint carries substantial weight loads during most of the day, in both static (e.g., standing and sitting) and dynamic (e.g., walking and running) conditions.

The hip joint is susceptible to a number of different pathologies. These pathologies can have both congenital and injury-related origins. In some cases, the pathology can be substantial at the outset. In other cases, the pathology may be minor at the outset but, if left untreated, may worsen over time. More particularly, in many cases, an existing pathology may be exacerbated over time by the dynamic nature of the hip joint and the substantial weight loads imposed on the hip joint.

The pathology may, either initially or thereafter, significantly interfere with patient comfort and lifestyle. In some cases, the pathology can be so severe as to require partial or total hip replacement. A number of procedures have been developed for treating hip pathologies short of such partial or total hip replacement, but these procedures are generally limited in scope due to the significant difficulties associated with treating a hip joint.

A better understanding of various hip joint pathologies, and also the current limitations associated with their treatment, can be gained from a more precise understanding of the anatomy of the hip joint.

### Anatomy of the Hip Joint

The hip joint is formed at the junction of the femur and the acetabulum. More particularly, and looking now at FIG. 2, the head of the femur is received in the acetabular cup, with a plurality of ligaments and other soft tissue serving to hold the bones in articulating relation.

More particularly, and looking now at FIG. 3, the femur is generally characterized by an elongated body terminating, at its top end, in an angled neck which supports a hemispherical head (also sometimes referred to as “the ball”). As seen in FIGS. 3 and 4, a large projection known as the greater trochanter protrudes laterally and posteriorly from the elongated body adjacent to the neck. A second, somewhat smaller projection known as the lesser trochanter protrudes medially and posteriorly from the elongated body adjacent to the neck. An intertrochanteric crest (FIGS. 3 and 4) extends along the periphery of the femur, between the greater trochanter and the lesser trochanter.

Looking next at FIG. 5, the hip socket is made up of three constituent bones: the ilium, the ischium and the pubis. These three bones cooperate with one another (they typically ossify into a single “hip bone” structure by about the age of 25) so as to form the acetabular cup. The acetabular cup receives the head of the femur.

Both the head of the femur and the acetabular cup are covered with a layer of articular cartilage which protects the underlying bone and facilitates motion. See FIG. 6.

Various ligaments and soft tissue serve to hold the ball of the femur in place within the acetabular cup. More particularly, and looking now at FIGS. 7 and 8, the ligamentum teres extends between the ball of the femur and the base of the acetabular cup. As seen in FIG. 9, a labrum is disposed about the perimeter of the acetabular cup. The labrum serves to increase the depth of the acetabular cup and effectively establishes a suction seal between the ball of the femur and the rim of the acetabular cup, thereby helping to hold the head of the femur in the acetabular cup. In addition to the foregoing, and looking now at FIG. 10, a fibrous capsule extends between the neck of the femur and the rim of the acetabular cup, effectively sealing off the ball-and-socket members of the hip joint from the remainder of the body. The foregoing structures (i.e., the ball-and-socket members, the ligamentum teres, the labrum and the fibrous capsule) are encompassed and reinforced by a set of three main ligaments (i.e., the iliofemoral ligament, the ischiofemoral ligament and the pubofemoral ligament) which extend between the femur and the perimeter of the hip socket. See FIGS. 11 and 12.

### Pathologies of the Hip Joint

As noted above, the hip joint is susceptible to a number of different pathologies. These pathologies can have both congenital and injury-related origins.

By way of example but not limitation, one important type of congenital pathology of the hip joint involves impingement between the neck of the femur and the rim of the acetabular cup. In some cases, and looking now at FIG. 13, this impingement can occur due to irregularities in the geometry of the femur. This type of impingement is sometimes referred to as cam-type femoroacetabular impingement (i.e., cam-type FAI). In other cases, and looking now at FIG. 14, the impingement can occur due to irregularities in the geometry of the acetabular cup. This latter type of impingement is sometimes referred to as pincer-type femoroacetabular impingement (i.e., a pincer-type FAI). Impingement can result in a reduced range of motion, substantial pain and, in some cases, significant deterioration of the hip joint.

By way of further example but not limitation, another important type of congenital pathology of the hip joint involves defects in the articular surface of the ball and/or in the articular surface of the acetabular cup. Defects of this type sometimes start out fairly small but can then increase in size over time, generally due to the dynamic nature of the hip joint and the weight loads imposed on the hip joint. Articular defects can result in substantial pain, induce and/or exacerbate arthritic conditions and, in some cases, cause significant deterioration of the hip joint.

By way of further example but not limitation, one important type of injury-related pathology of the hip joint involves trauma to the labrum. More particularly, in many cases, an accident or sports-related injury can result in the labrum being torn away from the rim of the acetabular cup, typically with a tear running through the body of the labrum. See FIG. 15. These types of injuries can be very painful for the patient and, if left untreated, can lead to substantial deterioration of the hip joint.

### The General Trend Toward Treating Joint Pathologies Using Minimally-Invasive, and Earlier, Interventions

The current trend in orthopedic surgery is to treat joint pathologies using minimally-invasive techniques. Such minimally-invasive, “keyhole” surgeries generally offer numerous advantages over traditional, “open” surgeries, including reduced trauma to the patient's tissue, less pain for the patient, faster recuperation times, etc.

By way of example but not limitation, it is common to re-attach ligaments in the shoulder joint using minimally-invasive, “keyhole” techniques which do not require “laying open” the capsule of the shoulder joint. By way of further example but not limitation, it is common to repair torn meniscal cartilage in the knee joint, and/or to replace ruptured ACL ligaments in the knee joint, using minimally-invasive techniques.

While such minimally-invasive approaches can require additional training on the part of the surgeon, such procedures generally offer substantial advantages for the patient and have now become the standard of care for many shoulder joint and knee joint pathologies.

In addition to the foregoing, in view of the inherent advantages and widespread availability of minimally-invasive approaches for treating pathologies of the shoulder joint and the knee joint, the current trend is to provide such treatment much earlier in the lifecycle of the pathology, so as to address patient pain as soon as possible and so as to minimize any exacerbation of the pathology itself. This is in marked contrast to traditional surgical practices, which have generally dictated postponing surgical procedures as long as possible so as to spare the patient from the substantial trauma generally associated with invasive surgery.

### Treatment for Pathologies of the Hip Joint

Unfortunately, minimally-invasive treatments for pathologies of the hip joint have lagged far behind minimally-invasive treatments for pathologies of the shoulder joint and the knee joint. This is generally due to (i) the geometry of the hip joint itself, and (ii) the nature of the pathologies which must typically be addressed in the hip joint.

More particularly, the hip joint is generally considered to be a “tight” joint, in the sense that there is relatively little room to maneuver within the confines of the hip joint itself. This is in marked contrast to the shoulder joint and the knee joint, which are generally considered to be relatively spacious joints, particularly when compared to the confines of the hip joint. As a result, it is generally relatively difficult for surgeons to perform minimally-invasive procedures on the hip joint.

Furthermore, the pathways for entering the interior of the hip joint (i.e., the pathways which exist between the bones of the patient) are generally much more constraining for the hip joint than for the shoulder joint or the knee joint. This limited access further complicates effectively performing minimally-invasive procedures on the hip joint.

In addition to the foregoing, the nature and location of the pathologies of the hip joint also complicate performing minimally-invasive procedures on the hip joint. By way of example but not limitation, consider a typical detachment of the labrum in the hip joint. In this situation, instruments must generally be introduced into the joint space using an angle of approach which is set at approximately a right angle to the angle of labrum re-attachment. This makes drilling into bone, for example, much more complex than where the angle of approach is effectively aligned with the angle of re-attachment, such as is frequently the case in the shoulder joint. Furthermore, the working space within the hip joint is typically extremely limited, further complicating repairs where the angle of approach is not aligned with the angle of re-attachment.

As a result of the foregoing, minimally-invasive hip joint procedures are still relatively difficult to perform and relatively uncommon in practice, and patients are typically forced to manage their hip joint pathologies for as long as possible, until a partial or total hip replacement can no longer be avoided, whereupon the procedure is generally done as a highly-invasive, open procedure, with all of the disadvantages associated with highly-invasive, open procedures.

As a result, there is a pressing need for improved methods and apparatus for reconstructing the hip joint.

### Arthroscopic Access to the Interior of the Hip Joint

Successful hip arthroscopy generally requires safe and reliable access to the interior of the hip joint. More particularly, successful hip arthroscopy generally requires the creation of a plurality of access portals which extend from the surface of the skin, through the underlying muscle tissue, through the capsule of the joint, and then down to the specific surgical site within the interior of the hip joint. Depending on the specific surgical site which is to be accessed within the interior of the hip joint, different anatomical pathways may be utilized for the access portals. By way of example but not limitation, one anatomical pathway may be used where a torn labrum is to be repaired, and another anatomical pathway may be used where the lesser trochanter must be addressed. And, in most cases, multiple access portals are generally required, with one access portal being used for visualization (i.e., to introduce an arthroscope into the interior of the hip joint), and with additional access portals being used to pass surgical instruments to and from the surgical site, etc.

However, the creation of access portals can be problematic. For one thing, the patient's anatomy (e.g., the particular location of bones, blood vessels, nerves, etc.) can greatly restrict the possible locations for the access portals. Furthermore, some hip structures (e.g., the articular cartilage on the femoral head, the articular cartilage on the acetabular cup, etc.) can be quite delicate, thereby requiring great precision when forming the access portal so as to avoid damaging these delicate anatomical structures. Additionally, some of the intervening tissue (e.g., the joint capsule) can be quite tough, thus requiring the generation of substantial forces in order to penetrate the tissue, and thereby presenting the possibility of accidental “plunging” as an access tool breaks through the tough intervening tissue. Such accidental plunging can result in inadvertent damage to any delicate joint structures (e.g., articular cartilage) located beneath the tough intervening tissue.

Due to the numerous difficulties and concerns associated with forming an access portal, surgeons have traditionally resorted to a multi-step procedure for forming an access portal.

More particularly, surgeons have traditionally first passed a small needle (sometimes referred to as an access needle) down to the interior of the hip joint. This is generally done by first using external anatomical landmarks and tactile feedback for needle insertion; then, as the sharp tip of the access needle penetrates the capsule of the hip joint and begins to approach delicate underlying structures (e.g., articular cartilage), fluoroscopy is used to carefully direct final needle placement. Inexperienced surgeons, or experienced surgeons dealing with particularly problematic cases, may also use fluoroscopy during the earlier stages of needle placement.

Next, a guidewire replaces the access needle and the tissue surrounding the guidewire is dilated (i.e., opened laterally) by passing a series of tissue dilators over the guidewire. These dilators can be devices sometimes referred to in the art as “switching sticks”. These dilators progressively increase in diameter so as to atraumatically laterally dilate the intervening tissue (e.g., the capsule) disposed between the skin and the interior of the joint. Depending on the extent of the dilation required and on the nature of the patient's tissue, anywhere from 1 to 3 dilators may be used in order to atraumatically achieve the degree of dilation desired.

Once the tissue surrounding the guidewire has been opened laterally to the extent desired, and once the last (i.e., largest) dilator has been withdrawn from the guidewire, a tubular liner (sometimes referred to as an access cannula) is then inserted over the guidewire. Alternatively, the access cannula is inserted over the dilator. This access cannula holds the incision open and provides a surgical pathway (or “corridor”) from the surface of the skin down to the interior of the hip joint, thereby enabling keyhole surgery to be performed on the hip joint.

Once the access cannula has been emplaced, the guidewire or dilator may be withdrawn, leaving the full diameter of the access cannula available for passing instruments and the like down to the surgical site. Alternatively, in many cases the guidewire may be left in place within the access cannula, with the guidewire thereafter being used for directing instruments down to the surgical site.

In an alternative multi-step procedure, a small access cannula may be inserted over the guidewire and into the joint space so as to at least partially dilate the tissue surrounding the guidewire. The small access cannula typically has an inner diameter ranging from approximately 4.0 mm to 5.5 mm, and preferably has an inner diameter ranging from approximately 4.5 mm to 5.0 mm. Small instruments can then be used through the small access cannula. However, since larger instruments (e.g. curved shavers and large burrs) will not fit through the small access cannula, the small access cannula must typically be removed at some point from the joint space and a larger access cannula passed over the guidewire and into the joint space. The passing of multiple access cannulas through the tissue layers between the skin and the joint compartment extends the duration of the procedure and may cause damage to the surrounding tissue layers.

Thus, as noted above, arthroscopic access to the interior of the hip joint generally requires the creation of an access portal into the interior of the hip joint. And as further noted above, the creation of an access portal into the interior of the hip joint in turn typically requires:

(i) passing an access needle from the surface of the skin down to the interior of the hip joint;

(ii) replacing the access needle with a guidewire; and

(iii) opening the tissue around the access needle in a lateral direction,

by either:
(a) passing a series of tissue dilators over the guidewire, with the tissue dilators progressively increasing in size so as to achieve the degree of dilation desired, then withdrawing the last (i.e., largest) dilator from the guidewire, and deploying an access cannula over the guidewire or dilator; or
(b) deploying a smaller access cannula over the guidewire, then inserting a switching stick, and delivering a larger access cannula through the tissue.

Thus, numerous steps are currently required in order to properly deploy an access cannula in the body. These multi-step processes require substantial effort on the part of the surgeon, increase the instrumentation necessary for the procedure, and extend the duration of the procedure.

On account of the foregoing, there is a substantial need for a simpler, faster and more convenient approach for creating an access portal to the interior of the hip joint.

### SUMMARY OF THE INVENTION

These and other objects of the present invention are addressed by the provision and use of a novel access cannula for accessing the interior of a hip joint. The novel access cannula is intended to provide atraumatic access to the interior of the hip joint without requiring the use of a series of dilators or cannulas. More particularly, the novel access cannula is constructed so that its distal end is laterally expandable, such that the access cannula can be inserted into the hip joint in a reduced-diameter configuration and then, once inside the hip joint, the access cannula can be laterally expanded so as to assume an enlarged-diameter configuration, whereby to atraumatically dilate the surrounding tissue. Thus, the laterally-expandable access cannula eliminates the need to pass a series of progressively-larger dilators or cannulas over the guidewire in order to dilate the tissue prior to installing the desired access cannula. In this way, the laterally-expandable access cannula provides a simpler, faster and more convenient approach for atraumatically providing an access portal to the interior of the hip joint.

In one preferred form of the invention, there is provided a laterally-expandable access cannula comprising:

an elongated body have a distal end, a proximal end and a lumen extending between the distal end and the proximal end, the distal end of the access cannula comprising a plurality of fingers tapering inwardly as they extend distally, and the elongated body having an internal thread extending along at least a portion of the length of the lumen; and

an inner sleeve disposed within the lumen of the elongated body, the inner sleeve having an external thread extending along at least a portion of its length, the external thread of the inner sleeve being in engagement with the internal thread of the elongated body, such that rotation of the inner sleeve causes the inner sleeve to move distally relative to the elongated body, whereby to cam open the plurality of fingers of the elongated body.

In another preferred form of the invention, there is provided a method for accessing a joint, the method comprising:

providing a laterally-expandable access cannula comprising:
an elongated body have a distal end, a proximal end and a lumen extending between the distal end and the proximal end, the distal end of the access cannula comprising a plurality of fingers tapering inwardly as they extend distally, and the elongated body having an internal thread extending along at least a portion of the length of the lumen; and
a inner sleeve disposed within the lumen of the elongated body, the inner sleeve having an external thread extending along at least a portion of its length, the external thread of the inner sleeve being in engagement with the internal thread of the elongated body, such that rotation of the inner sleeve causes the inner sleeve to move distally relative to the elongated body, whereby to cam open the plurality of fingers of the elongated body;

inserting the laterally-expandable access cannula into tissue; and

advancing the inner sleeve within the lumen of the elongated body so as to cam open the fingers of the elongated body.

In another preferred form of the invention, there is provided an access cannula comprising an elongated body having a distal end, a proximal end and a lumen extending between the distal end and the proximal end, the distal end of the access cannula comprising a plurality of flexible fingers tapering inwardly as they extend distally, with a membrane extending about the plurality of flexible fingers so that the distal end of the access cannula normally has reduced inner and outer diameters relative to the remainder of the access cannula.

In another form of the invention, there is provided an obturator comprising an elongated body having a distal end, a proximal end and a lumen extending between the distal end and the proximal end, the distal end of the obturator comprising a conical structure followed by a plurality of spaced-apart fins.

And in another form of the invention, there is provided an access cannula system comprising:

an access cannula comprising an elongated body having a distal end, a proximal end and a lumen extending between the distal end and the proximal end, the distal end of the access cannula comprising a plurality of flexible fingers tapering inwardly as they extend distally, with a membrane extending about the plurality of flexible fingers so that the distal end of the access cannula normally has reduced inner and outer diameters relative to the remainder of the access cannula; and

an obturator comprising an elongated body having a distal end, a proximal end and a lumen extending between the distal end and the proximal end, the distal end of the obturator comprising a conical structure followed by a plurality of spaced-apart fins.

And in another form of the invention, there is provided a method for accessing a joint, comprising:

providing an access cannula system comprising:
an access cannula comprising an elongated body having a distal end, a proximal end and a lumen extending between the distal end and the proximal end, the distal end of the access cannula comprising a plurality of flexible fingers tapering inwardly as they extend distally, with an membrane extending about the plurality of flexible fingers so that the distal end of the access cannula normally has reduced inner and outer diameters relative to the remainder of the access cannula;
an obturator comprising an elongated body having a distal end, a proximal end and a lumen extending between the distal end and the proximal end, the distal end of the obturator comprising a conical structure followed by a plurality of spaced-apart fins; and
a guidewire;

passing the guidewire from outside the body, through intervening tissue and into the joint;

inserting the obturator within the lumen of the access cannula so that the fins of the obturator are disposed between the flexible fingers of the access cannula, with the membrane holding the distal end of the access cannula in a reduced-diameter configuration;

passing the access cannula/obturator assembly down the guidewire so that the access cannula/obturator assembly extends through the intervening tissue; and

rotating the obturator within the lumen of the access cannula so that the fins of the obturator engage and cam outboard the flexible fingers of the access cannula, with the membrane yielding so as to permit the distal end of the access cannula to enter an expanded-diameter configuration, whereby to dilate adjacent tissue.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIGS. 1A-1D are schematic views showing various hip joint motions;

FIG. 2 is a schematic view showing the ball-and-socket nature of the hip joint;

FIG. 3 is a schematic view showing the femur;

FIG. 4 is a schematic view showing the upper end of the femur;

FIG. 5 is a schematic view showing the acetabulum;

FIG. 6 is a schematic view showing the articular cartilage of the hip joint;

FIGS. 7 and 8 are schematic views showing the ligamentum teres of the hip joint;

FIG. 9 is a schematic view showing the labrum of the hip joint;

FIG. 10 is a schematic view showing the capsule of the hip joint;

FIGS. 11 and 12 are schematic views showing the three main ligaments (i.e., the iliofemoral ligament, the ischiofemoral ligament and the pubofemoral ligament) which extend between the femur and the perimeter of the acetabular cup;

FIG. 13 is a schematic view showing cam-type femoroacetabular impingement;

FIG. 14 is a schematic view showing pincer-type femoroacetabular impingement;

FIG. 15 is a schematic view showing a labral tear;

FIGS. 16-18A are schematic views showing a novel laterally-expandable access cannula formed in accordance with the present invention, wherein the laterally-expandable access cannula comprises a threaded inner sleeve adapted to move longitudinally relative to the access cannula so as to selectively laterally expand the distal end of the access cannula;

FIGS. 19-23 are schematic views showing how longitudinal movement of the threaded inner sleeve laterally expands the distal end of the access cannula;

FIGS. 24-26 are schematic views showing how a keyed driver may be used to rotate the threaded inner sleeve so as to longitudinally advance the threaded inner sleeve within the access cannula, whereby to laterally expand the distal end of the access cannula;

FIGS. 27-29 are schematic views showing another form of keyed driver which may be used to rotate the threaded inner sleeve so as to longitudinally advance the threaded inner sleeve within the access cannula, whereby to laterally expand the distal end of the access cannula;

FIG. 30 is a schematic view showing a novel access cannula sytem formed in accordance with the present invention, wherein the access cannula system comprises an access cannula, an obturator and a guidewire;

FIG. 31 is a schematic view showing further details of the access cannula shown in FIG. 30;

FIG. 32 is a schematic view showing the distal end of the access cannula shown in FIG. 30, with the access cannula's membrane removed for clarity of illustration;

FIG. 33 is a schematic side view, partially in section, showing the distal end of the access cannula shown in FIG. 30;

FIG. 34 is a schematic side view, partially in section, showing the proximal end of the access cannula shown in FIG. 30;

FIG. 35 is a schematic view showing further details of the obturator shown in FIG. 30;

FIG. 36 is a schematic view showing the distal end of the obturator;

FIG. 37 is a schematic side view, partially in section, showing the obturator extending through the access cannula, wherein the obturator is disposed so that the access cannula is in its reduced-diameter configuration;

FIG. 38 is a schematic front view showing the obturator extending through the access cannula, wherein the obturator is disposed so that the access cannula is in its reduced-diameter configuration;

FIG. 39 is a schematic view showing the obturator extending through the access cannula, with the access cannula's membrane removed for clarity of illustration, and wherein the obturator is disposed so that the access cannula is in its reduced-diameter configuration;

FIG. 40 is a schematic view showing the obturator extending through the access cannula, with the access cannula's membrane removed for clarity of illustration, and wherein the obturator is disposed so that the access cannula is in its enlarged-diameter configuration;

FIG. 41 is a schematic view showing the obturator extending through the access cannula, wherein the obturator is disposed so that the access cannula is in its enlarged-diameter configuration; and

FIGS. 42-49 are a series of schematic views showing the novel access cannula system of FIG. 30 being used to form an access portal to the interior of the hip joint.

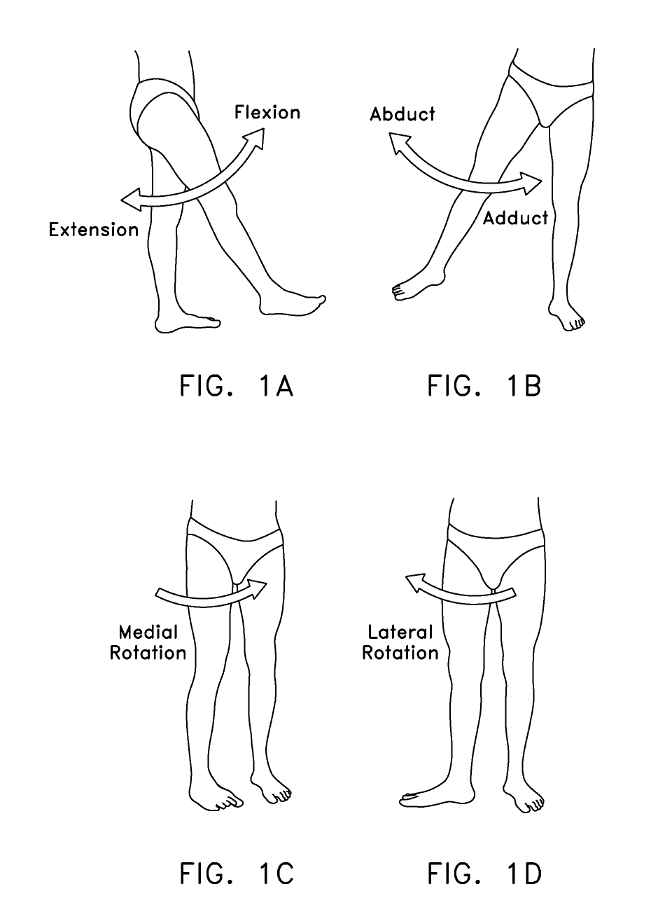

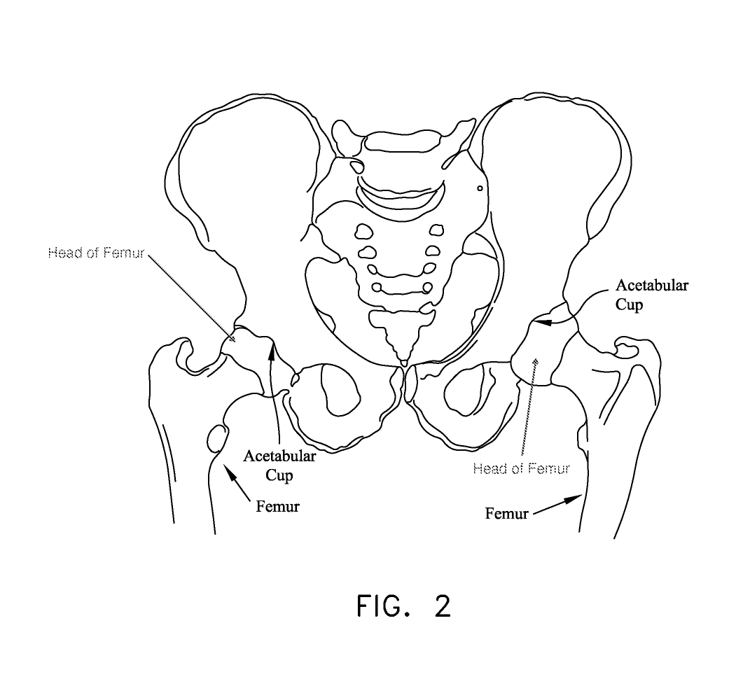

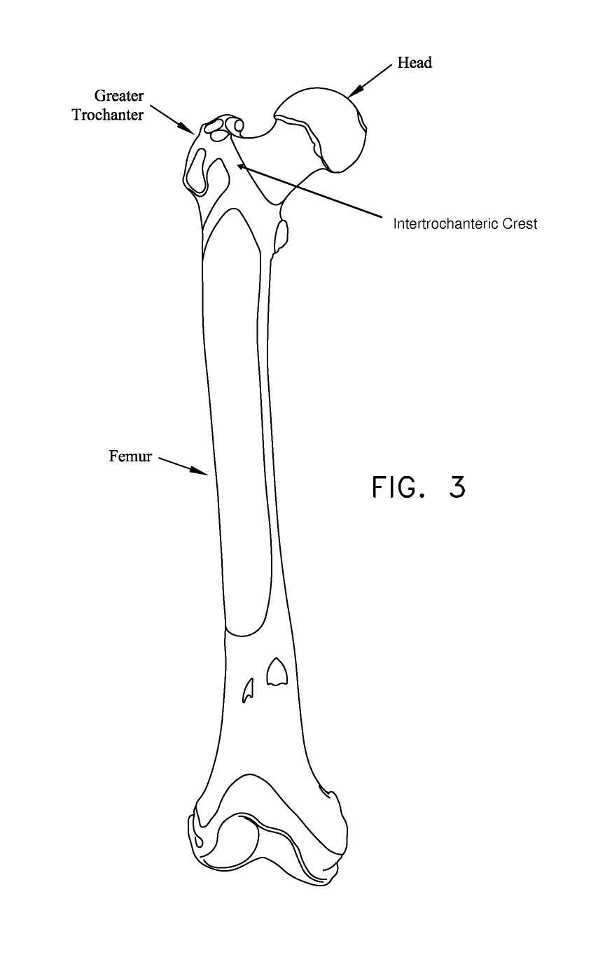

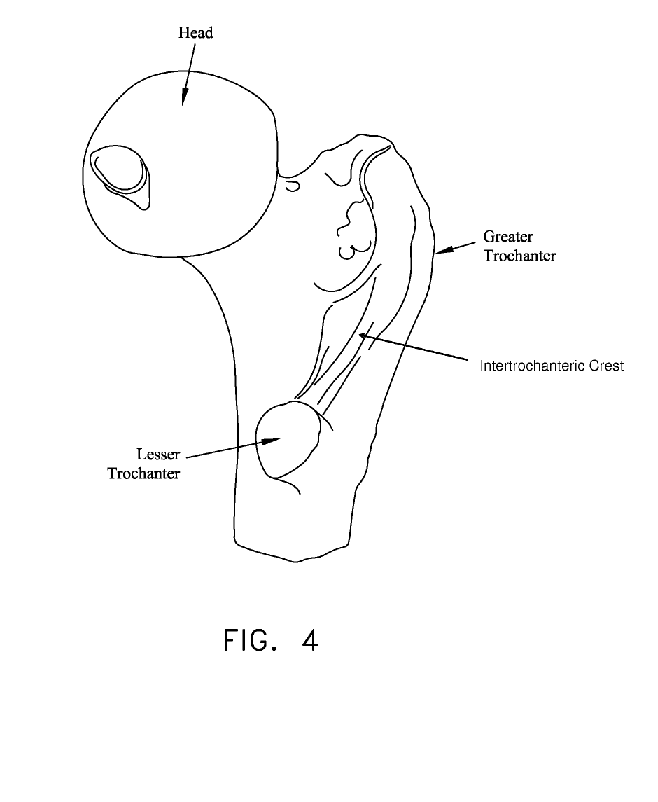

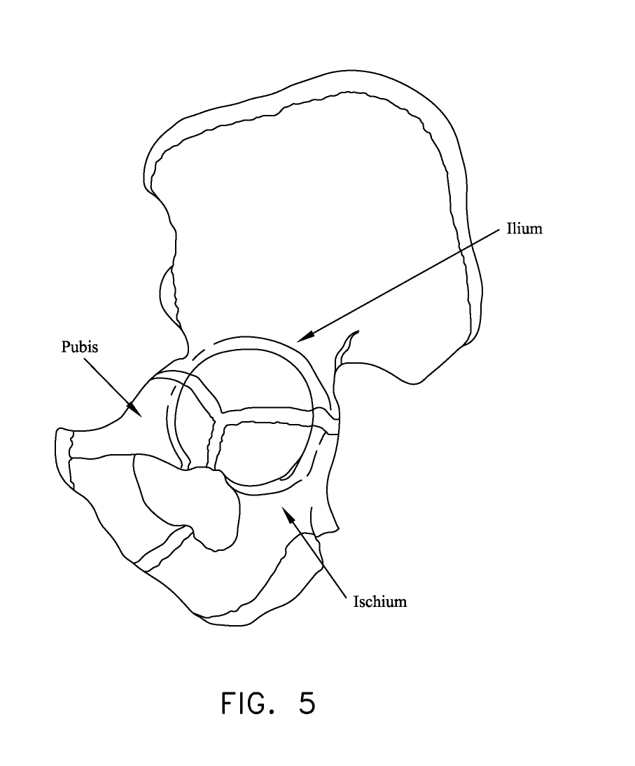

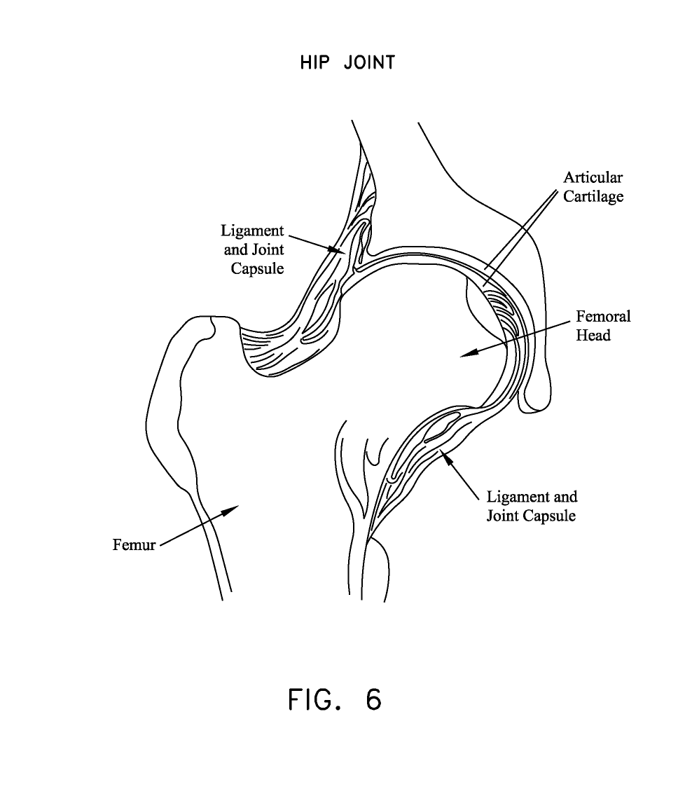

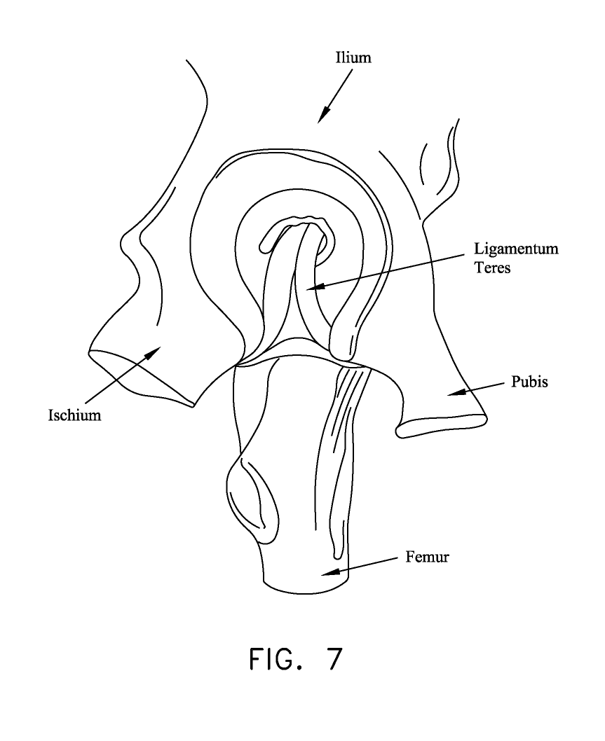

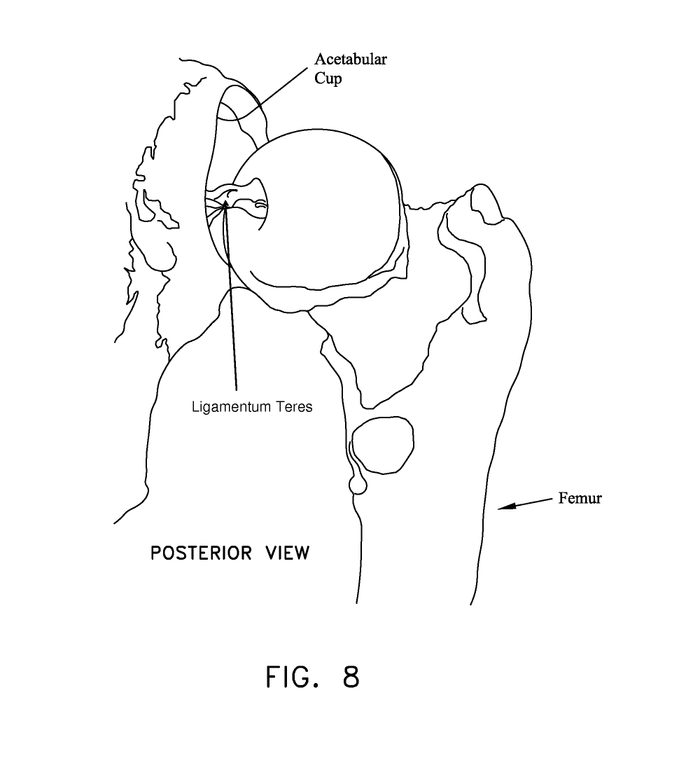

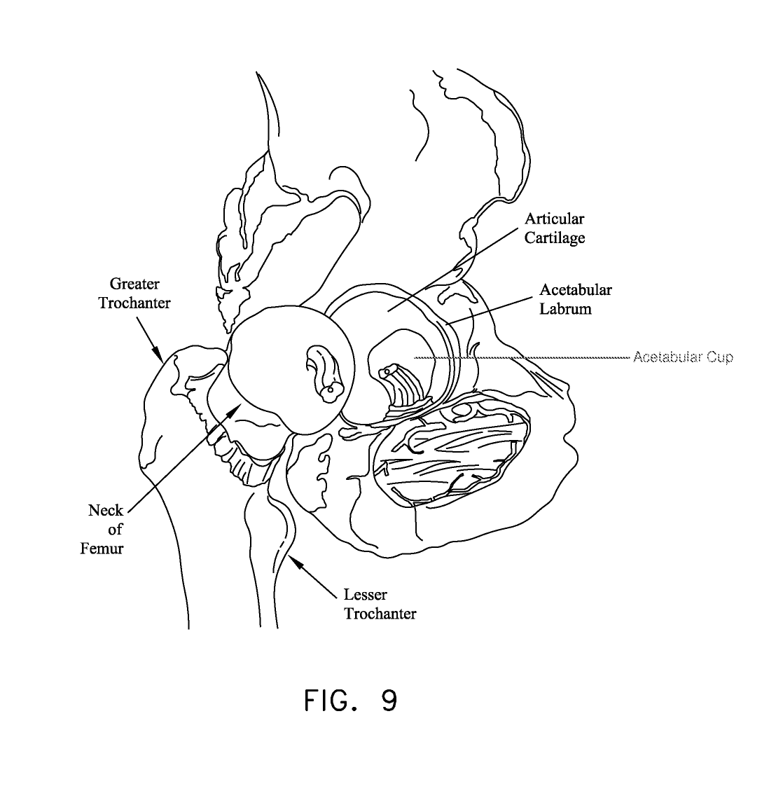

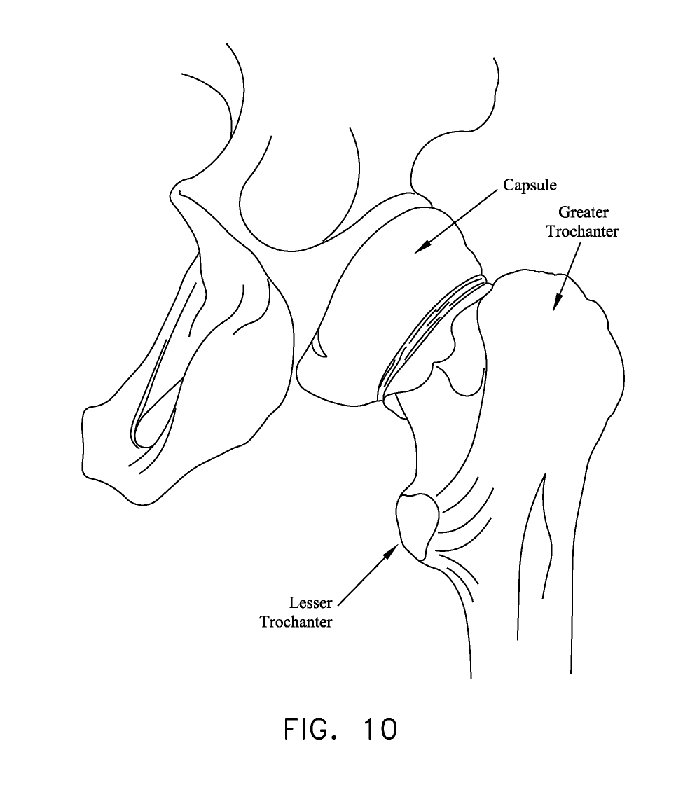

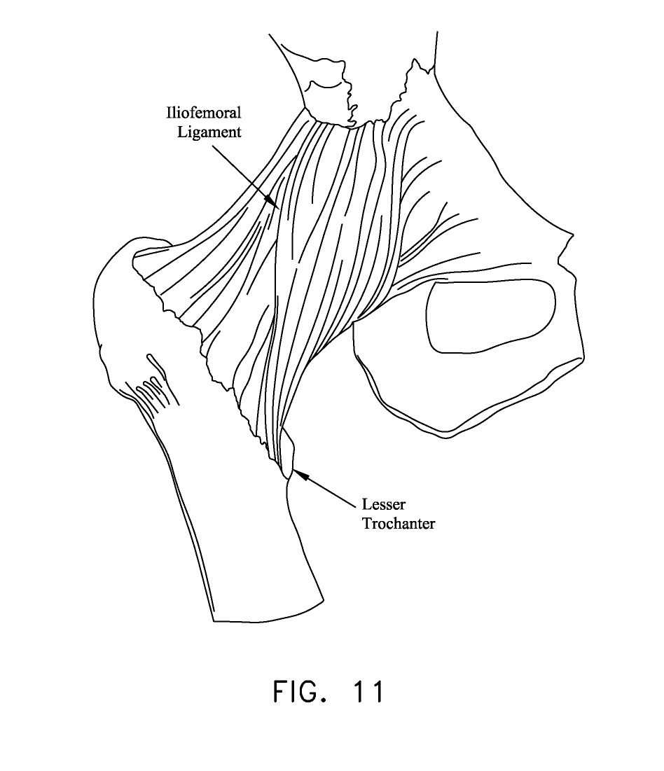

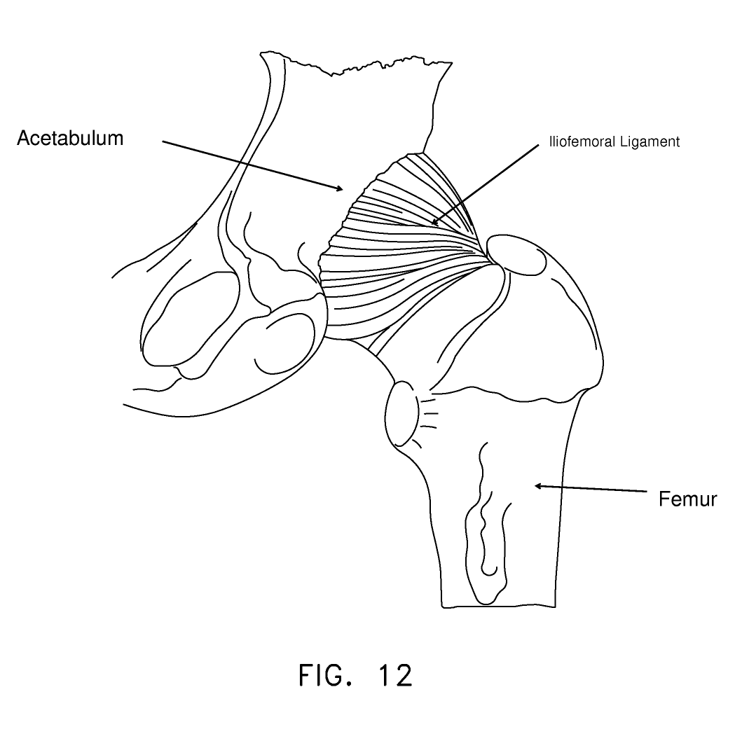

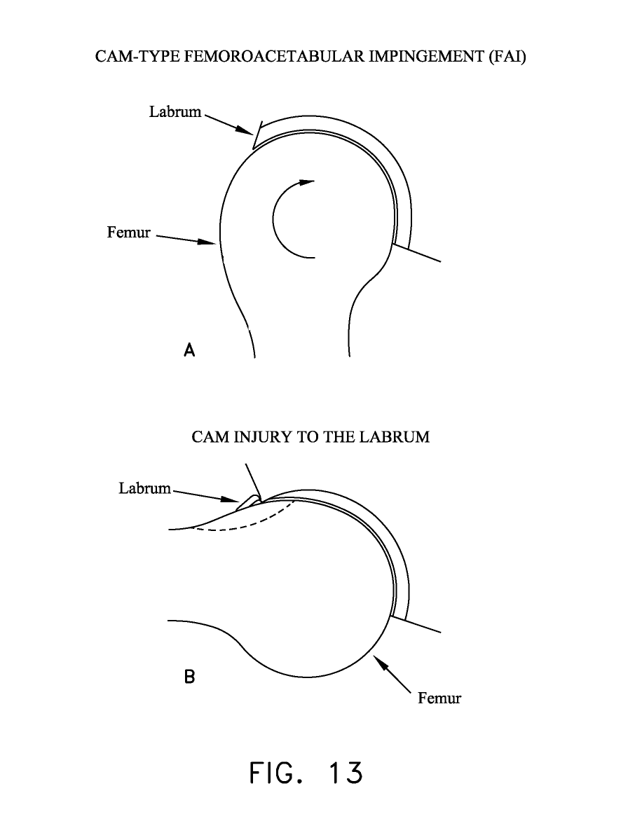

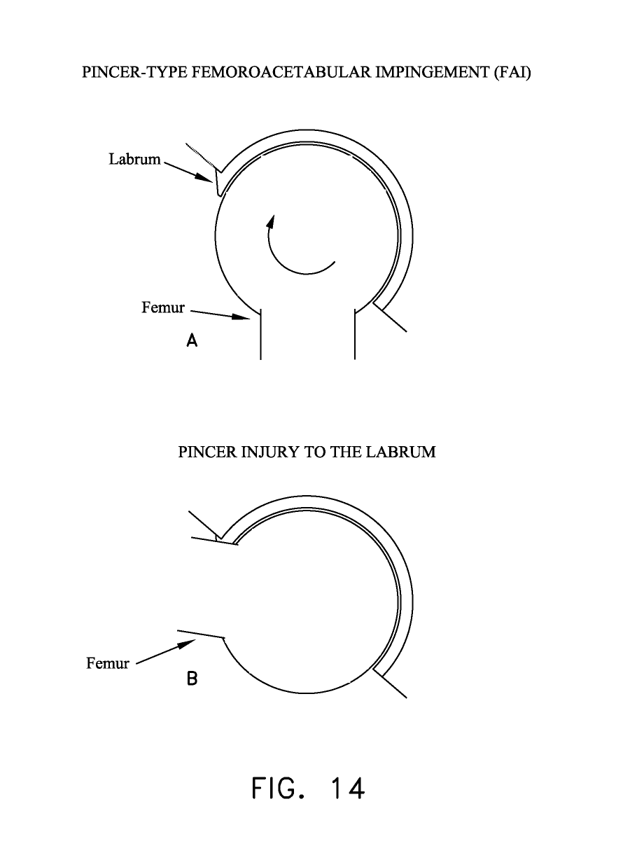

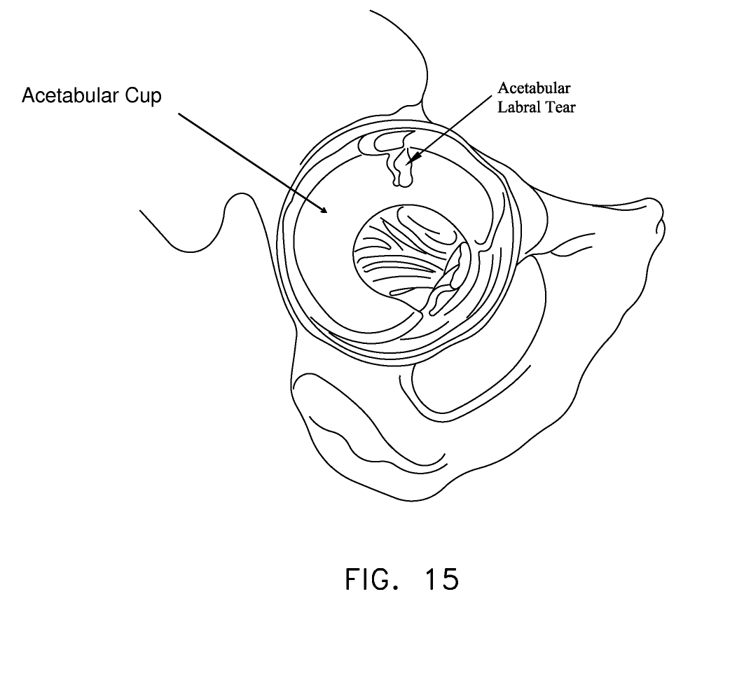

### DETAILED DESCRIPTION OF THE INVENTION

The present invention is directed to a novel laterally-expandable access cannula which provides a simpler, faster and more convenient approach for atraumatically providing an access portal to the interior of the hip joint.

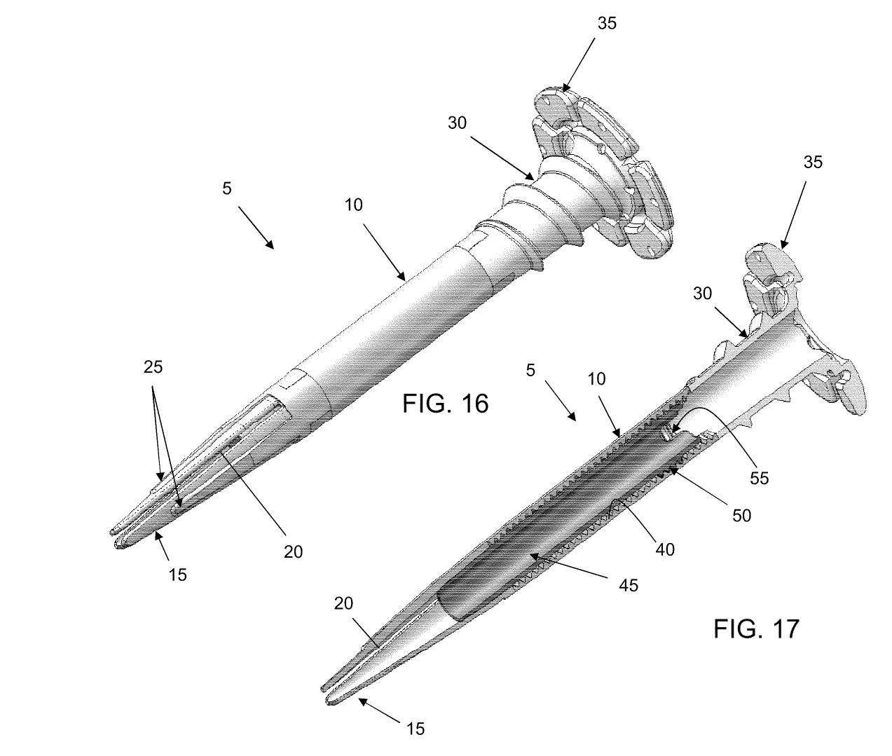

More particularly, and looking now at FIGS. 16-18A, there is shown a novel, laterally-expandable access cannula 5 formed in accordance with the present invention. Laterally-expandable access cannula 5 generally comprises a tubular body 10 having a tapered distal end 15 (FIGS. 16 and 17) characterized by a plurality of longitudinally-extending slits 20, whereby to define a plurality of distally-converging fingers 25. Preferably, the distalmost tips of fingers 25 are slightly spaced from one another, so as to accommodate a guidewire therebetween, as will hereinafter be discussed in further detail.

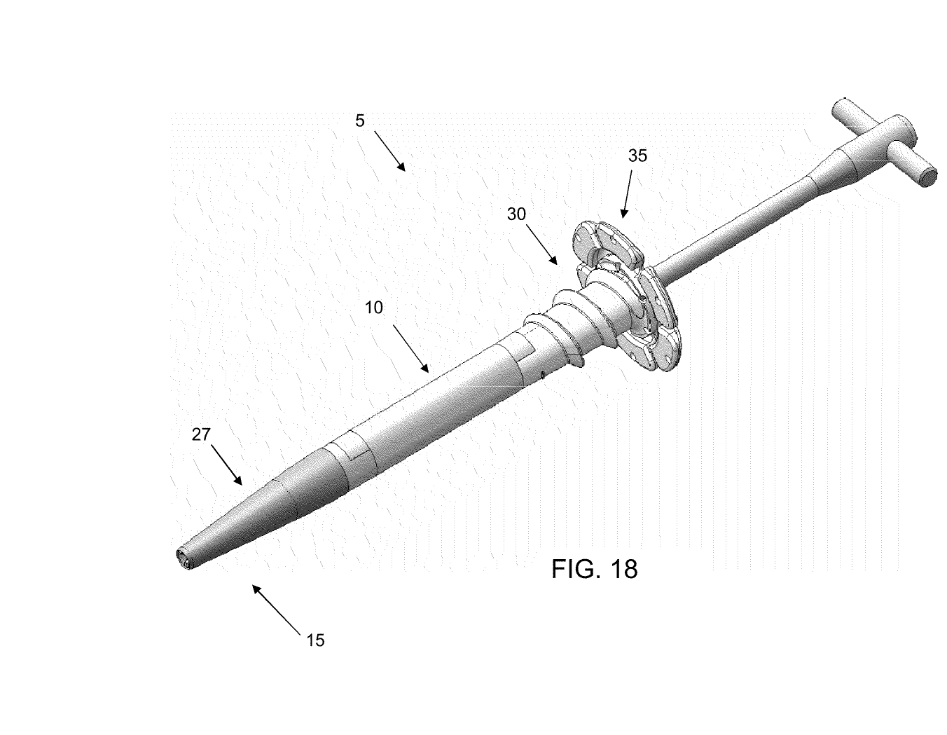

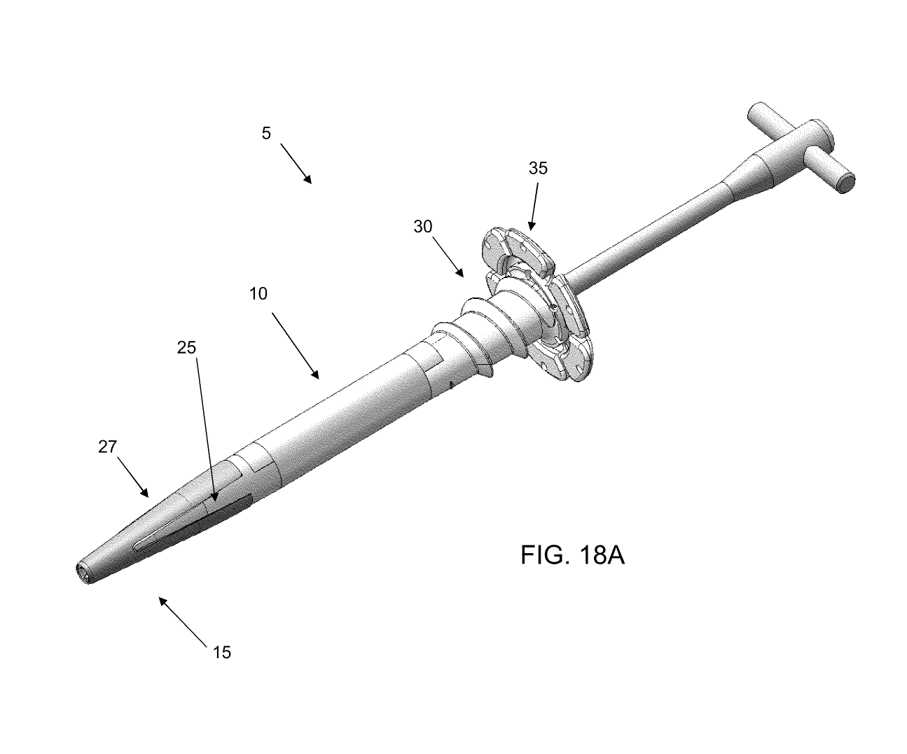

In one embodiment, a membrane 27 may form a boot over the fingers (FIG. 18). Alternatively, membrane 27 may span the spaces between flexible fingers 25 (FIG. 18A). The membrane can stretch or otherwise yield so that flexible fingers 25 can move radially outwardly when appropriately urged in that direction. The membrane may be a separate component which is joined to the cannula or the membrane may be formed integral with the cannula, such as with an insert-molding process. The membrane may be formed out of an elastomeric material such as silicone rubber, or urethane, or it may be formed out of a semi-elastomeric or non-elastomeric material, e.g., folded-up high strength polyethelene (PE) or polyethylene terephthalate (PET). It can be compliant, semi-compliant or non-compliant. Preferably, the membrane is of a low durometer, e.g., approximately 50 shore A.

The proximal end 30 of laterally-expanding access cannula 5 terminates in an enlarged flange 35. The interior side wall of laterally-expandable access cannula 5 includes screw threads 40.

An inner sleeve 45 is disposed within the interior of laterally-expandable access cannula 5, with exterior screw threads 50 of inner sleeve 45 engaging interior screw threads 40 of laterally-expandable access cannula 5.

As a result of this construction, rotation of inner sleeve 45 causes inner sleeve 45 to move longitudinally within laterally-expandable access cannula 5. To this end, one or more recesses 55 may be provided on the proximal end of threaded inner sleeve 45 in order to facilitate rotation of inner sleeve 45, as will hereinafter be discussed in further detail below.

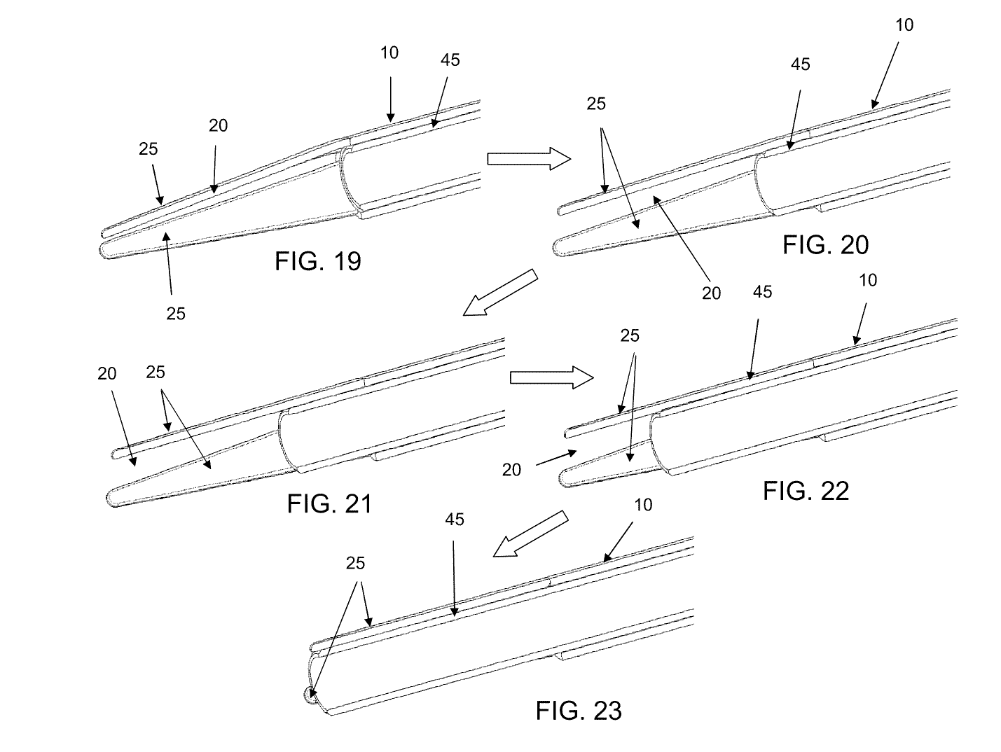

In view of the foregoing construction, and looking now at FIGS. 16-23, it will be seen that when inner sleeve 45 is in its proximal position (FIGS. 16, 17 and 19), the distal end of inner sleeve 45 is located proximal to fingers 25 at the distal end of access cannula 5, so that fingers 25 are free to converge at the distal end of the access cannula. However, as seen in FIGS. 20-23, advancement of inner sleeve 45 in a distal direction causes the distal end of inner sleeve 45 to cam open fingers 25, whereby to laterally expand the distal end of the access cannula.

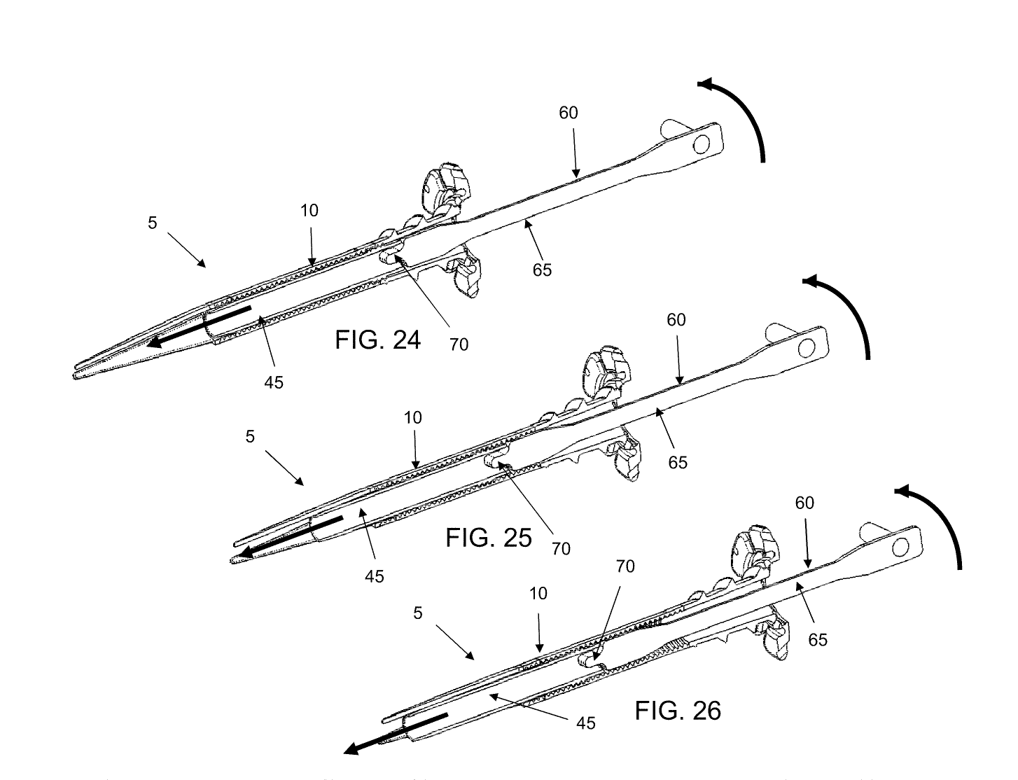

As seen in FIGS. 24-26, a keyed driver 60 may be used to reach down into the interior of access cannula 5 in order to rotate inner sleeve 45. To this end, keyed driver 60 generally comprises an elongated shaft 65 terminating in one or more keys 70. Keys 70 are received within recesses 55 of inner sleeve 45, whereby to transmit rotation of keyed driver 60 to inner sleeve 45. The keyed driver may be integrated into a delivery obturator so that the obturator performs both the function of inserting the cannula into the tissue and rotating the inner sleeve 45.

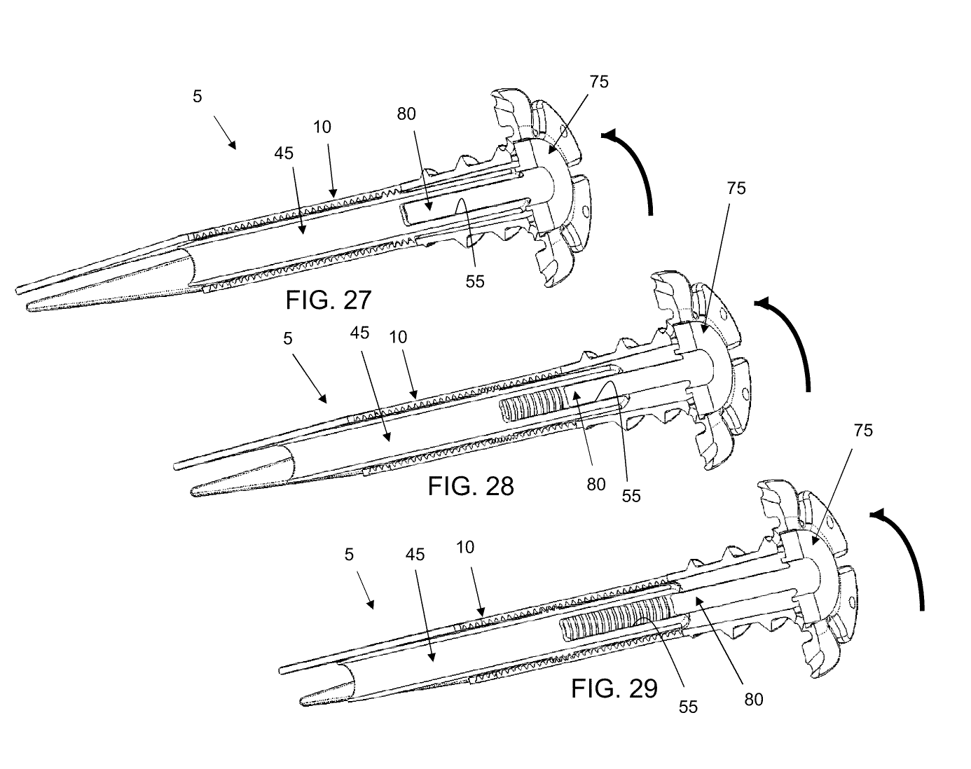

Alternatively, and looking now at FIGS. 27-29, a keyed driver 75 may be used to rotate inner sleeve 45. More particularly, keyed driver 75 is rotatably mounted to access cannula 5. One or more keys 80 are formed at the distal end of keyed driver 75. Keys 80 are configured to be received in recesses 55 of inner sleeve 45, whereby to transmit rotation of keyed driver 75 to threaded inner sleeve 45. Preferably, inner sleeve 45 comprises a plurality of recesses 55, and keyed driver 75 comprises a plurality of keys 80, whereby to provide a splined drive mechanism for transferring rotational motion of keyed driver 75 into longitudinal movement of inner sleeve 45.

Screw threads 40 of access cannula 5 and screw threads 50 of inner sleeve 45 can be single lead or multiple lead, such as six thread lead. Screw threads 40 and 50 can be a short pitch or long pitch.

Alternatively, inner sleeve 45 can be advanced distally in access cannula 5 and locked into place without the use of threads. For example, inner sleeve 45 and access cannula 5 can have features that form a bayonet style mechanism. Alternatively, the proximal end of inner sleeve 45 can extend beyond the proximal end of access cannula 5 and the two proximal ends can lock together when inner sleeve 45 is advanced distally.

During use, a guidewire is first passed through the surface tissue of the patient, through the intervening muscle tissue, through the capsule and then into the interior space of the hip joint. Next, with access cannula 5 in its reduced-diameter configuration, access cannula 5 is passed coaxially over the guidewire, with the guidewire being disposed within the interior of the access cannula, and into the tissue. Access cannula 5 is advanced until the distal tip of the access cannula protrudes through the capsule and into the interior of the hip joint. Then the guidewire is removed.

Next, threaded internal sleeve 45 is rotated relative to the access cannula (via keyed driver 60 or via keyed driver 75) so as to force cannula fingers 25 outwardly. This action causes the tissue surrounding the distal end of the access cannula to be atraumatically dilated.

When the access cannula is to be removed or re-positioned, threaded internal sleeve 45 may be rotated (via keyed driver 60 or via keyed driver 75) so as to return the access cannula to its reduced-diameter configuration, and then the access cannula may be removed from, or re-positioned within, the surgical site.

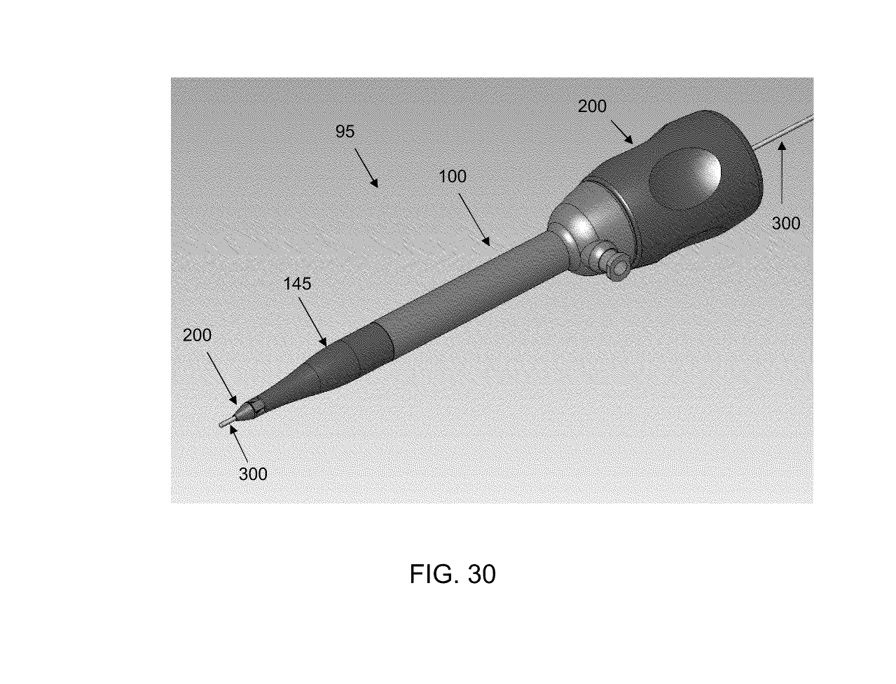

In another form of the invention, and looking now at FIG. 30, there is shown a novel laterally-expandable access cannula system 95 formed in accordance with the present invention. Access cannula system 95 generally comprises an access cannula 100 for providing a lined access corridor to a remote surgical site, an obturator 200 for disposition in access cannula 100 during cannula insertion so as to prevent tissue coring, and a guidewire 300 to facilitate proper placement of access cannula 100 (and obturator 200) relative to the surgical site. Significantly, access cannula 100 is constructed so that its distal end is laterally expandable, such that the access cannula can be inserted into the hip joint in a reduced-diameter configuration and then, once inside the hip joint, the access cannula can be laterally expanded as instruments whose diameters are larger than the normal (i.e., relaxed) internal diameter of the cannula (i.e., the distance between the fingers) are passed through the cannula. The cannula, then, assumes the diameter of the instrument, whereby the adjacent tissue is atraumatically dilated.

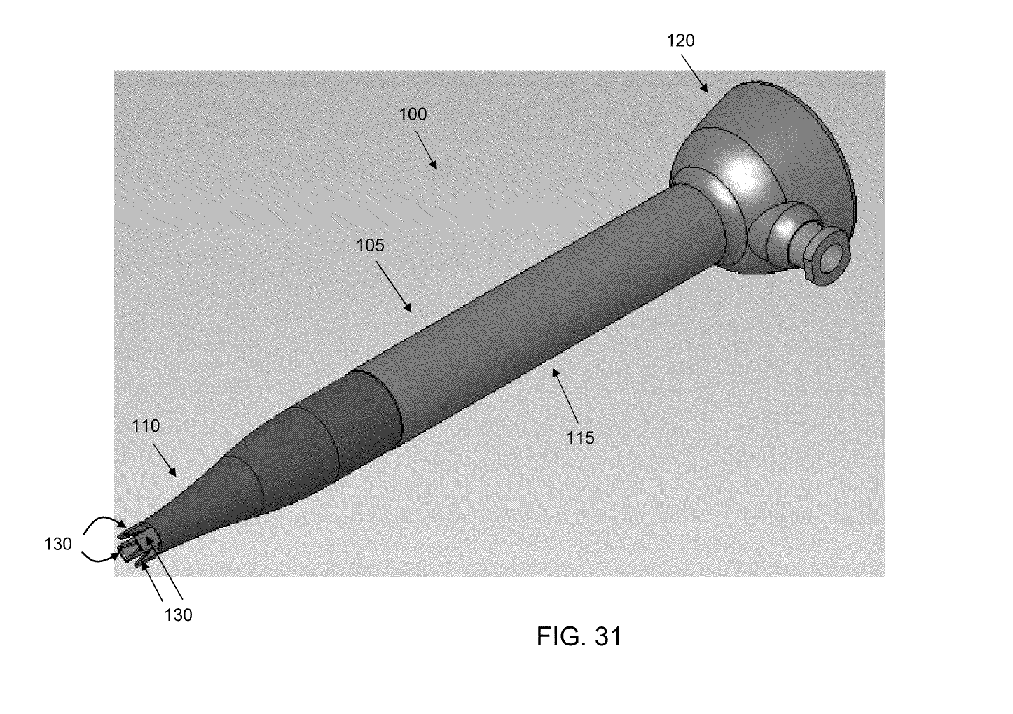

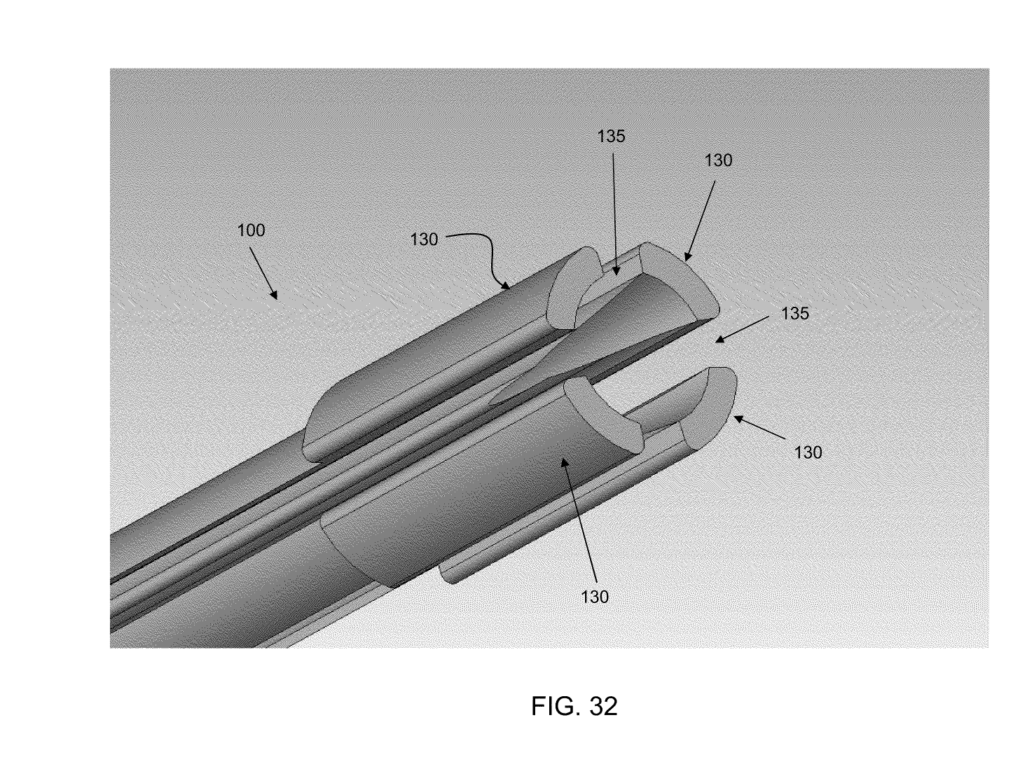

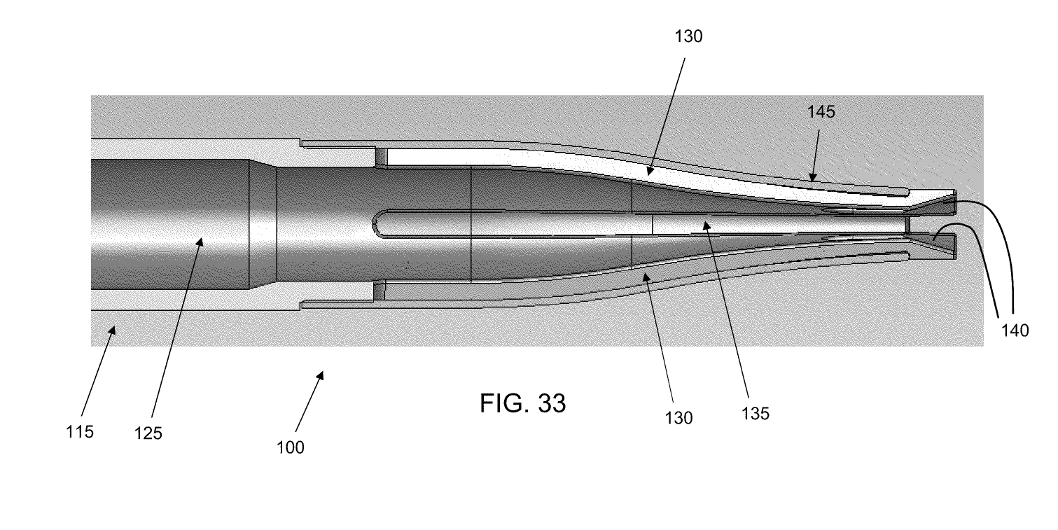

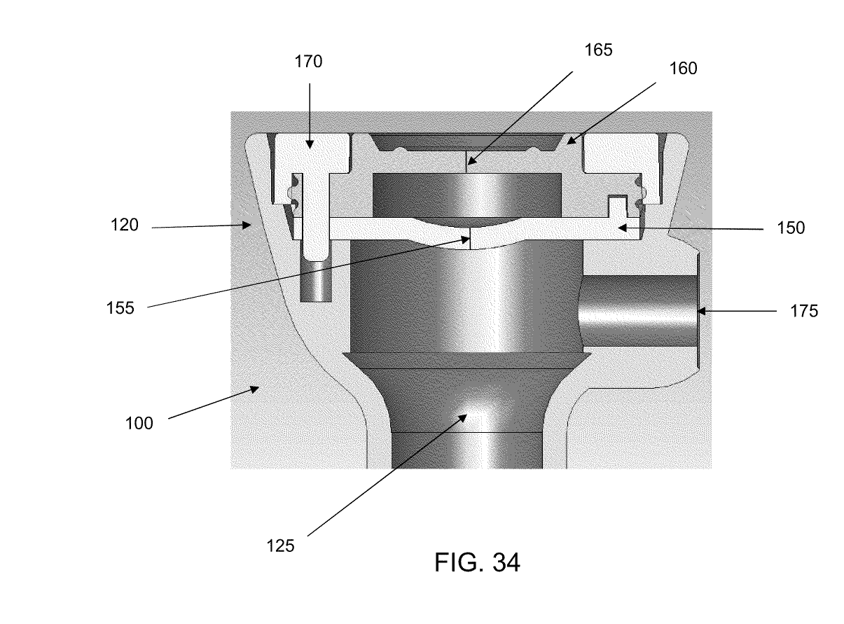

Looking next at FIGS. 30-34, access cannula 100 generally comprises a cannula body 105 having a distal end 110, an intermediate portion 115 and a proximal end 120. A lumen 125 (FIGS. 33 and 34) extends between distal end 110 and proximal end 120.

Distal end 110 comprises a plurality of flexible fingers 130 separated by slits 135. Flexible fingers 130 taper inwardly as they extend distally so that the distalmost portion of the cannula body has reduced inner and outer diameters relative to intermediate portion 115 of the cannula body (FIG. 33). At the distalmost end of flexible fingers 130, the inner surfaces of the flexible fingers can have an internal bevel 140. A membrane 145 is mounted coaxially over flexible fingers 130. Membrane 145 serves to bias flexible fingers 130 radially inwardly so that the fingers normally assume the configuration shown in FIG. 33. However, and as will hereinafter be discussed in further detail, membrane 145 can stretch so that flexible fingers 130 can move radially outwardly when appropriately urged in that direction. In this embodiment, membrane 145 provides a smooth exterior surface for distal end 110 of access cannula 100 which minimizes trauma to surrounding tissue as the access cannula is manipulated in the tissue.

In an alternative embodiment, membrane 145 does not bias flexible fingers 130 inwardly but is in a substantially natural, unstressed state. In yet another embodiment, flexible fingers 130 have an inward or outward bias that is resisted by membrane 145.

Membrane 145 may be a separate component which is joined to distal end 110 of access cannula 100, or membrane 145 may be formed integral with distal end 110 of access cannula 100, such as with an insert-molding process. Membrane 145 may be formed out of an elastomeric material such as silicone rubber, polyethylene terephthalate (PET) or urethane, or it may be formed out of a non-elastomeric material, e.g., folded-up high strength polyethylene (PE). It can be compliant, semi-compliant or non-compliant.

The proximal end of cannula body 105 is provided with a distal seal 150 (FIG. 34) having a slit 155 therein, and a proximal seal 160 having a slit 165 therein. Distal seal 150 and proximal seal 160 are held in place within cannula body 105 by a cap 170. Distal seal 150 and proximal seal 160 serve to close off lumen 125 to fluid flow, while their slits 155, 165 permit an instrument to be passed through the seals. A luer lock port 175, which may be sealed off by a luer lock cap (not shown), permits fluid to bypass seals 155, 165.

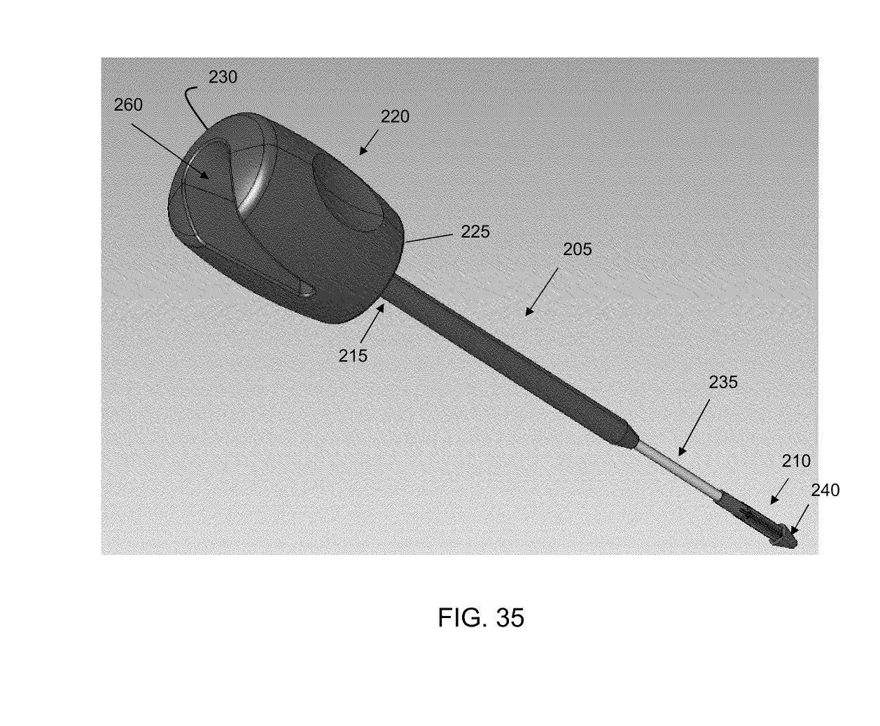

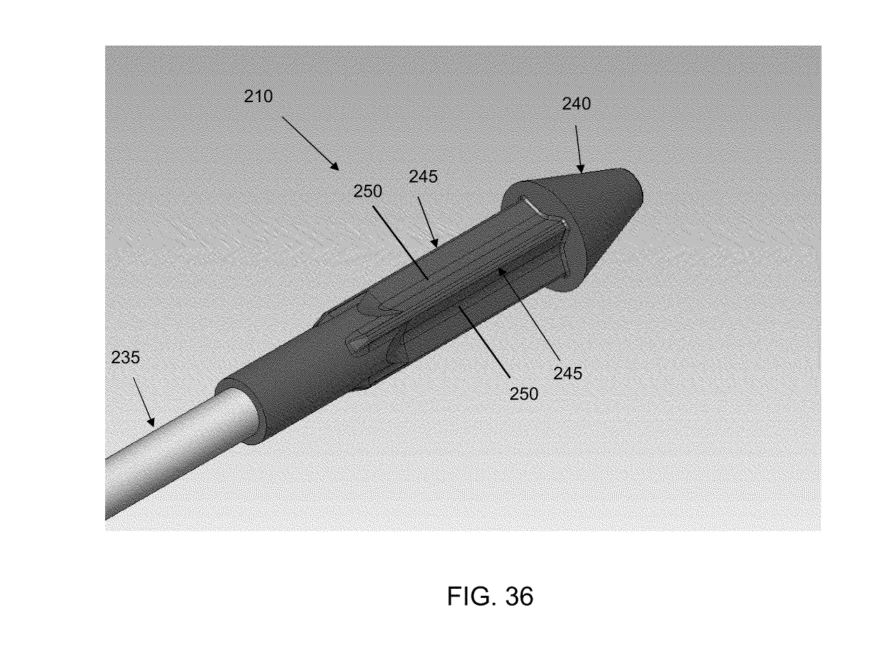

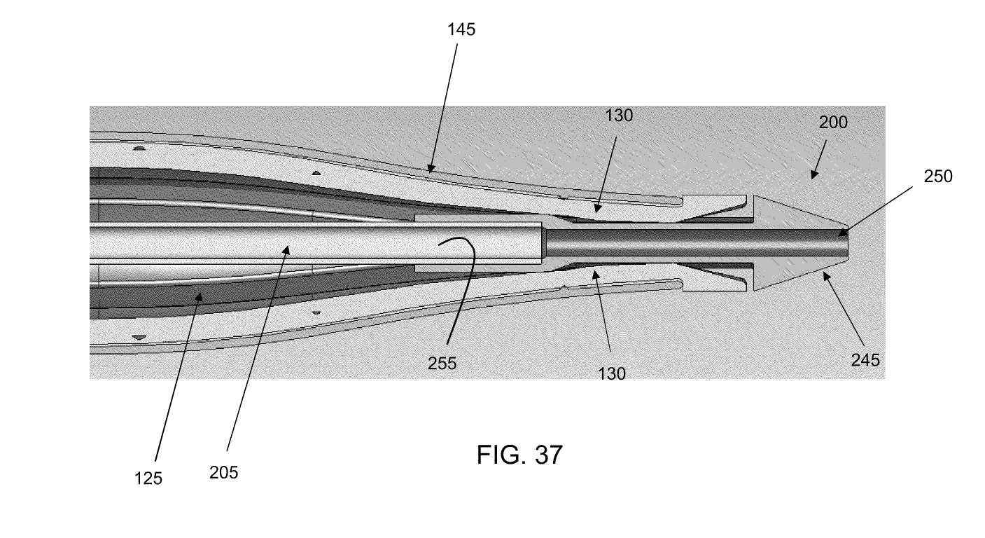

Looking next at FIGS. 30, 35 and 36, obturator 200 generally comprises a shaft 205 having a distal end 210 and a proximal end 215. Obturator 200 also comprises a handle 220 having a distal end 225 and a proximal end 230. Proximal end 215 of shaft 205 is connected to distal end 225 of handle 220. If desired, an intermediate portion 235 of shaft 205 may be formed out of a stiffer material than the remainder of obturator 200, e.g., intermediate portion 235 may be formed out of stainless steel while the remainder of obturator 200 may be formed out of plastic. Distal end 210 of shaft 205 comprises a conical tip 240 followed by fins 245. Recesses 250 are provided between fins 245. A lumen 255 (not shown in FIGS. 35 and 36, but shown in FIG. 37) extends the length of obturator 200. A slot 260 extends radially through handle 220 and communicates with lumen 255, as will hereinafter be discussed in further detail.

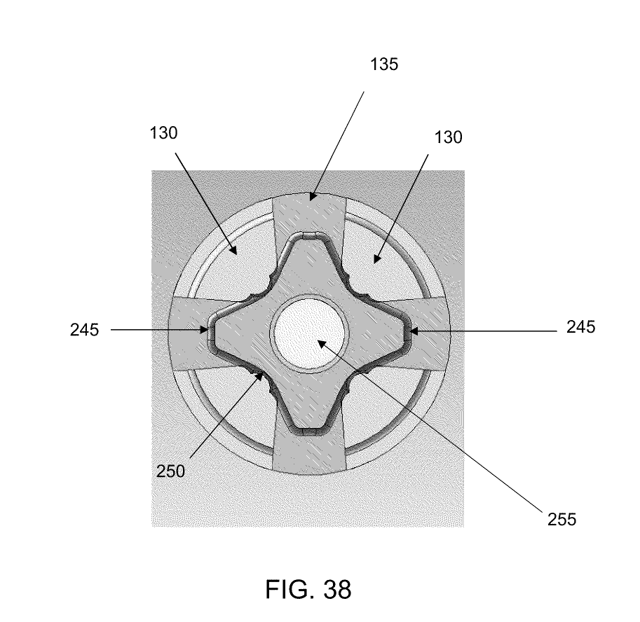

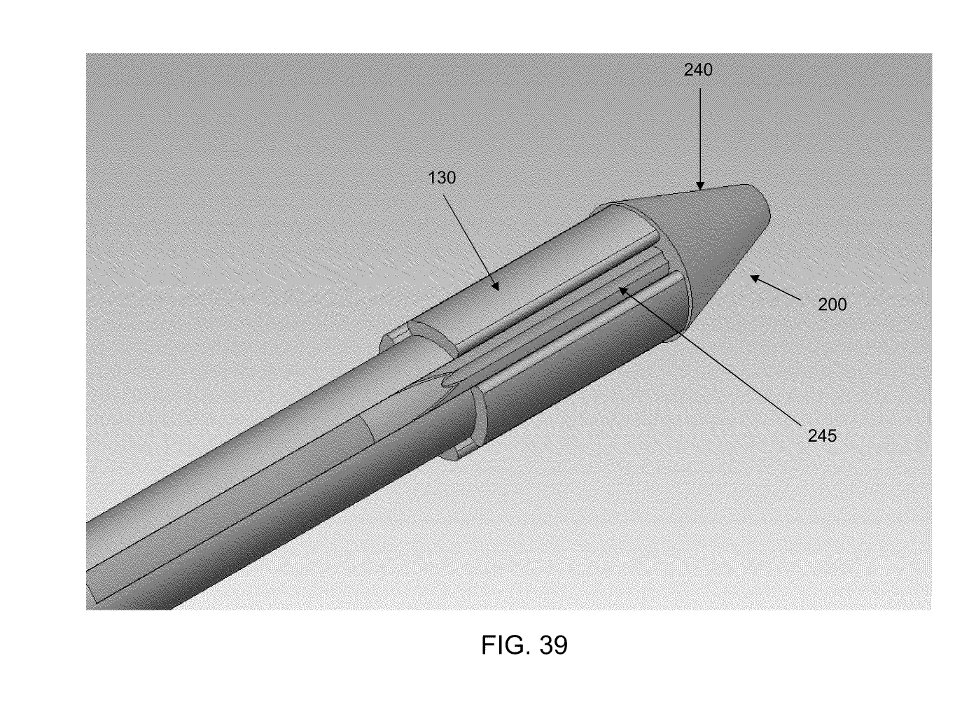

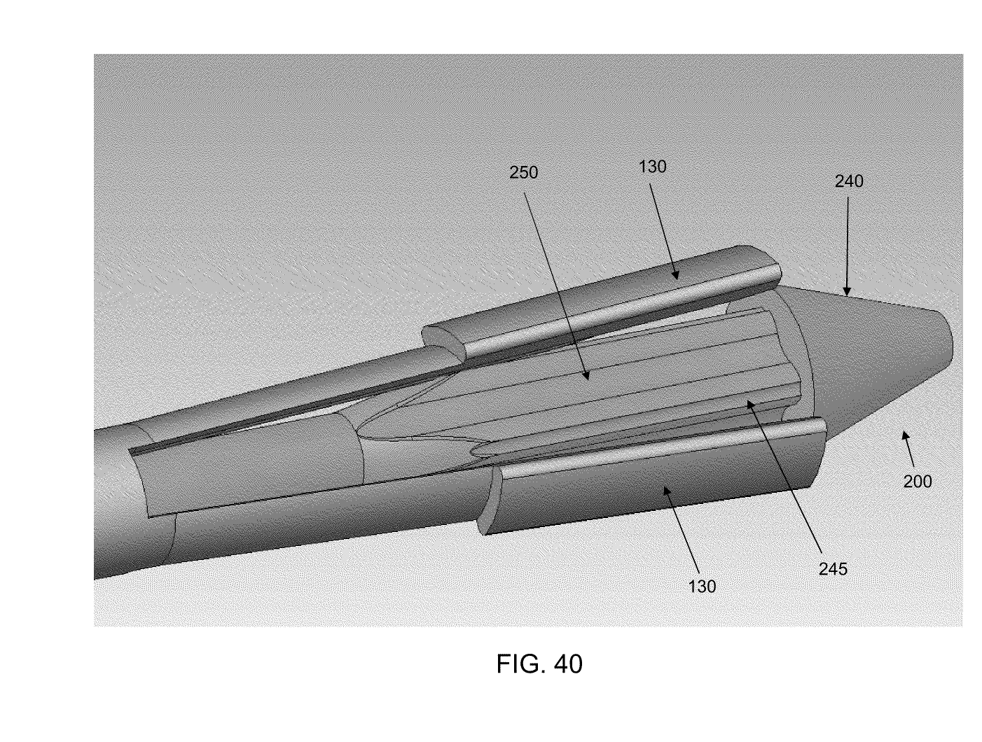

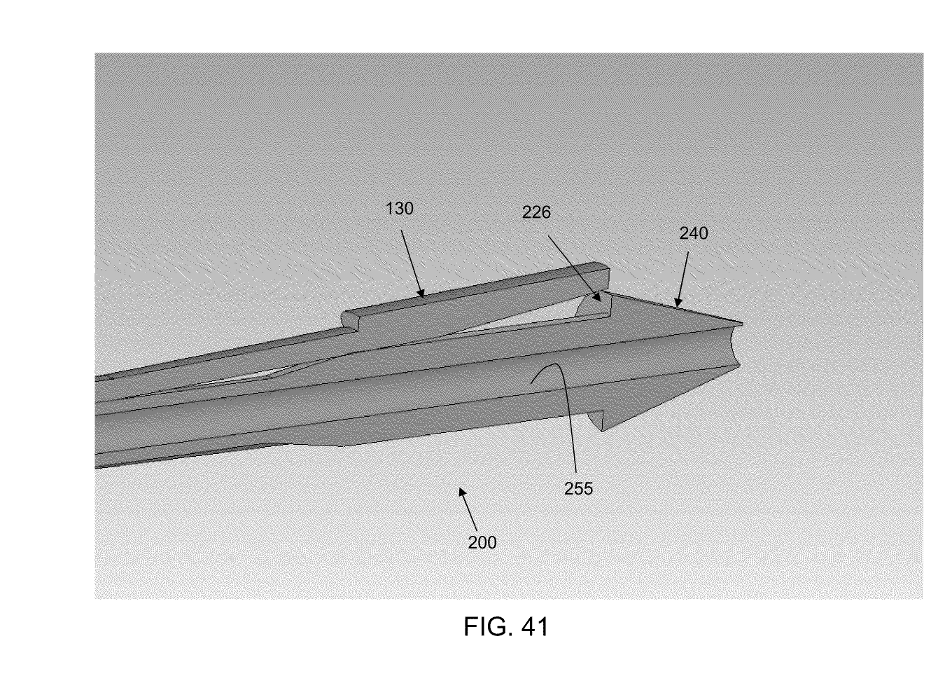

Looking now at FIGS. 30 and 37-41, obturator 200 is sized so that its shaft 205 can be positioned within lumen 125 of access cannula 100, with the obturator's fins 245 being positioned within slots 135 of flexible fingers 130 (FIGS. 37-39) when the obturator's handle 220 is in engagement with the cannula's proximal end 120. In this position, flexible fingers 130 are in their relaxed state, biased inwardly under the influence of membrane 145, so that flexible fingers 130 are disposed in obturator recesses 250, thus providing an autraumatic profile for joint entry. However, when obturator 200 is rotated within cannula 100, fins 245 engage flexible fingers 130 and effectively cam those flexible fingers outboard, against the bias of membrane 145, whereby to increase the diameter of the distal tip of cannula 100 and thus allow the obturator to be removed (FIGS. 40 and 41).

In an alternative embodiment, bevel 140 (FIG. 33) cams flexible fingers 130 outward as obturator is retracted. The proximal side 226 of obturator tip 240 may have a corresponding bevel which further facilitates the camming action (FIG. 41).

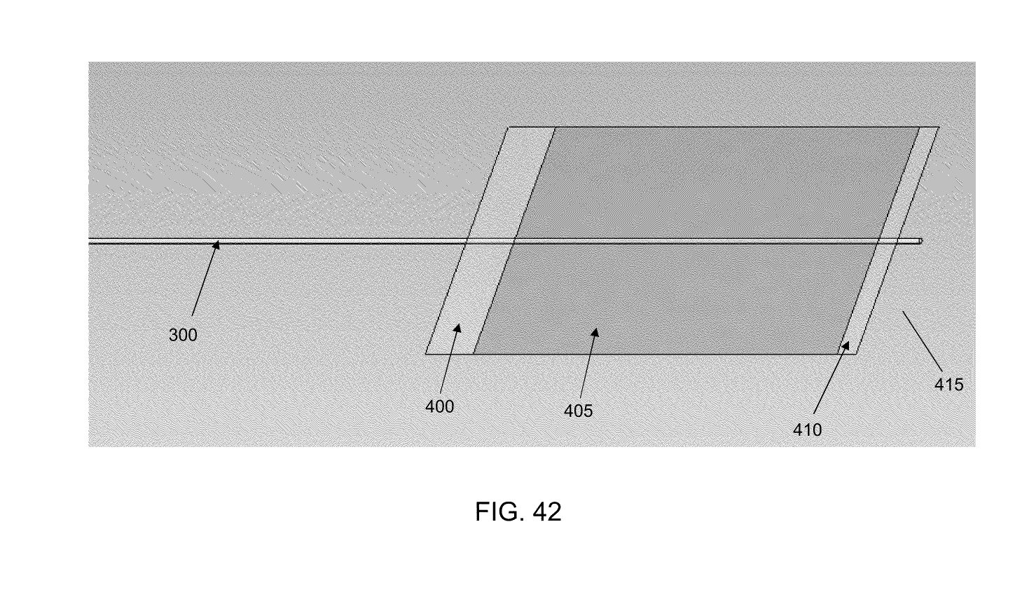

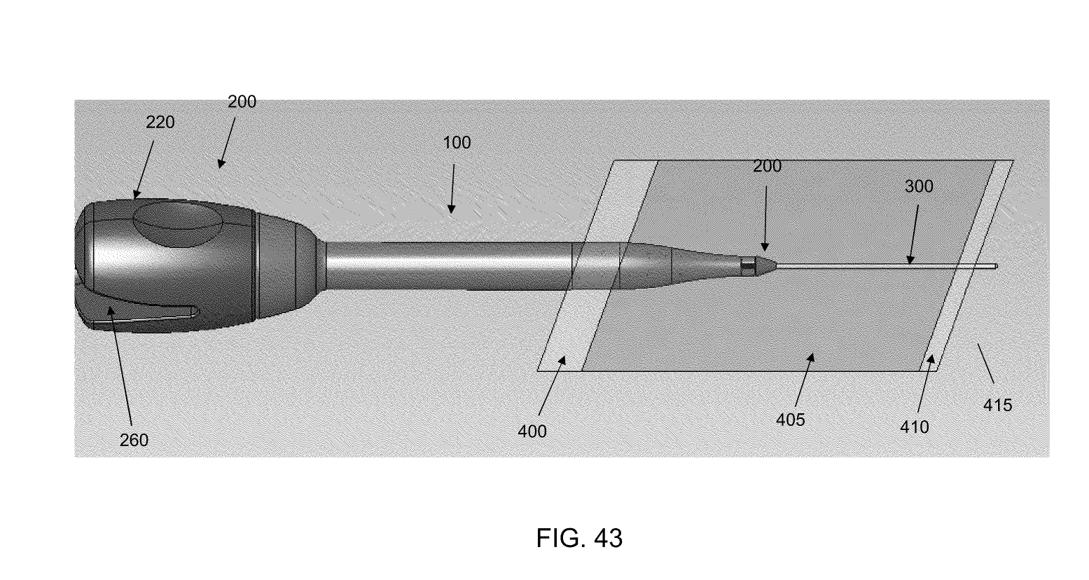

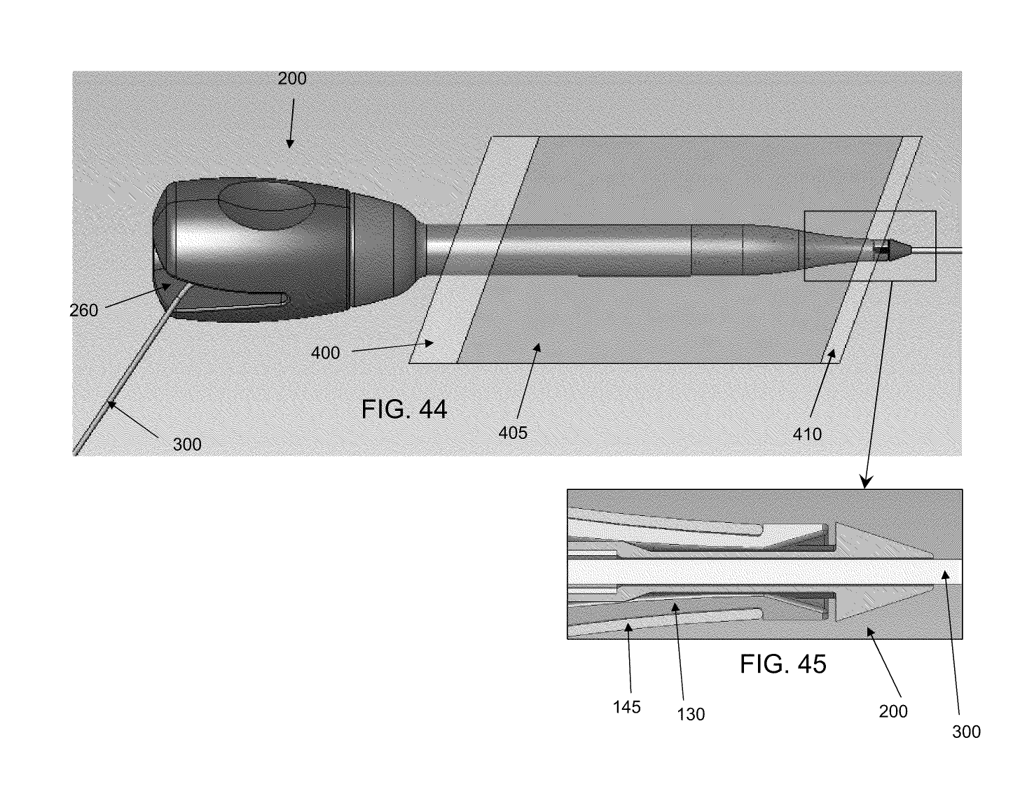

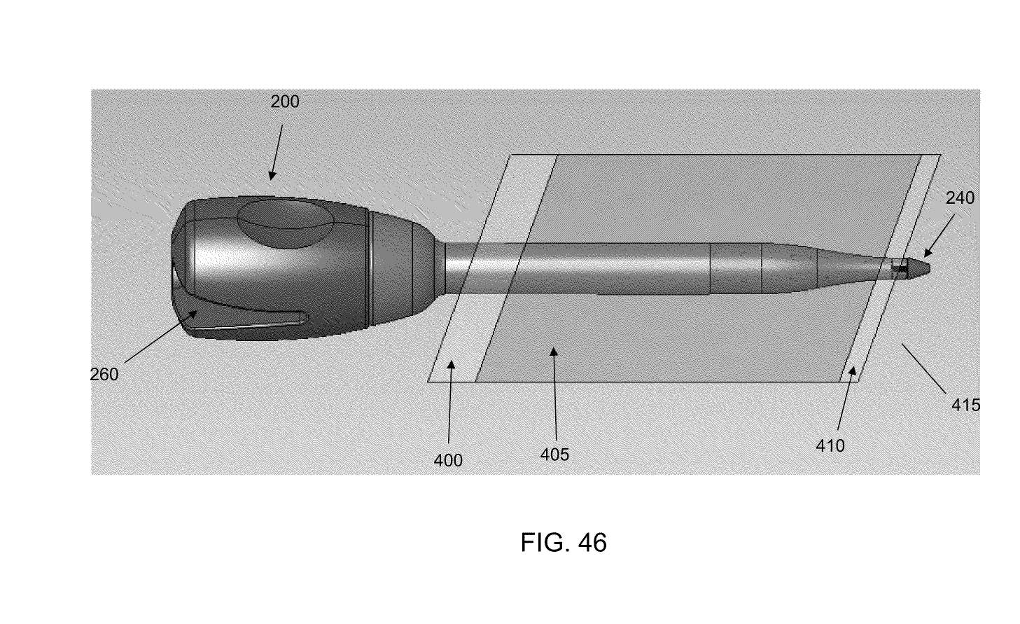

During use, and looking next at FIG. 42, a guidewire 300 is first passed through surface tissue 400, through the intervening tissue 405, through the capsule 410 and then into the interior space 415 of the hip joint. Next, obturator 200 is inserted into access cannula 100 so that the distal end of the obturator extends beyond the distal end of the access cannula and the obturator's fins 245 are positioned within slots 135 of flexible fingers 130, and so that the distal end of the obturator's handle engages the proximal end of the access cannula. At this point the access cannula is in its reduced-diameter configuration (FIGS. 37-39). With the access cannula in this reduced-diameter configuration, the access cannula/obturator assembly is passed coaxially over guidewire 300 (FIG. 43), with the guidewire being disposed within lumen 255 of the obturator, and into the tissue. Obturator handle 220 is used to push the assembly through the tissue (FIGS. 43-45). During this advancement, guidewire 300 may be bent sideways through slot 260 of obturator handle 220 so as to move the guidewire radially, away from the proximal end of the obturator handle, thereby facilitating pushing on proximal end 230 of obturator handle 220. The access cannula/obturator assembly is advanced until the distal tip of the assembly protrudes through the capsule and into the interior of the hip joint (FIG. 44). Then guidewire 300 is removed (FIG. 46).

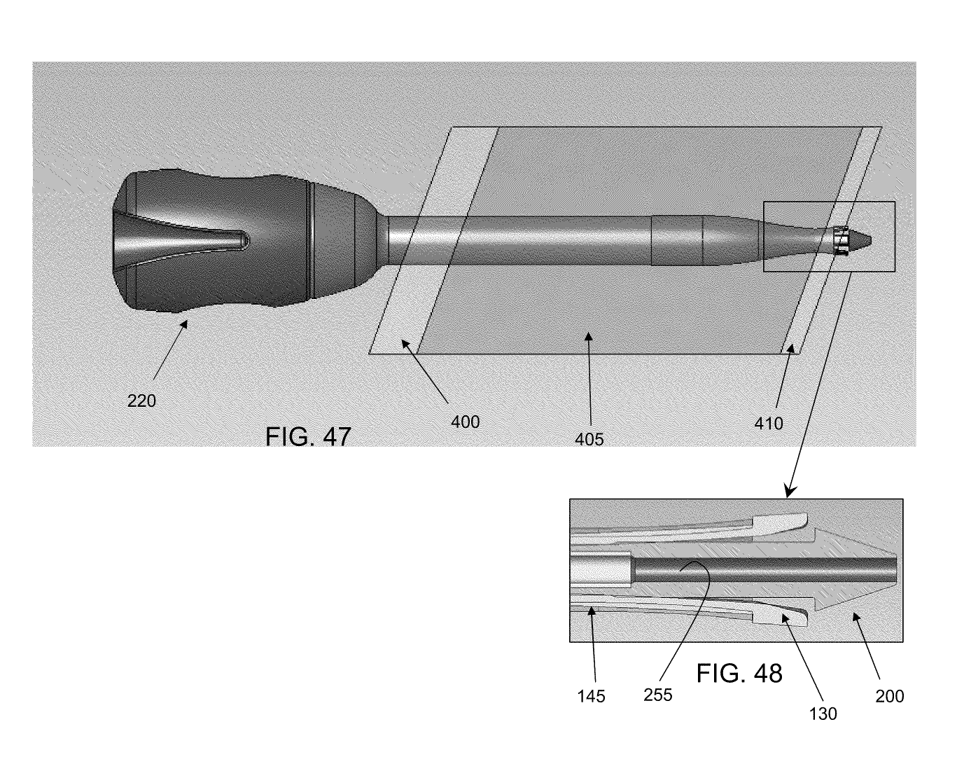

Next, obturator 200 is rotated relative to access cannula 100 so as to force cannula fingers 130 outwardly, against the force of membrane 145, (FIGS. 47 and 48). This action allows the obturator to be removed from the access cannula.

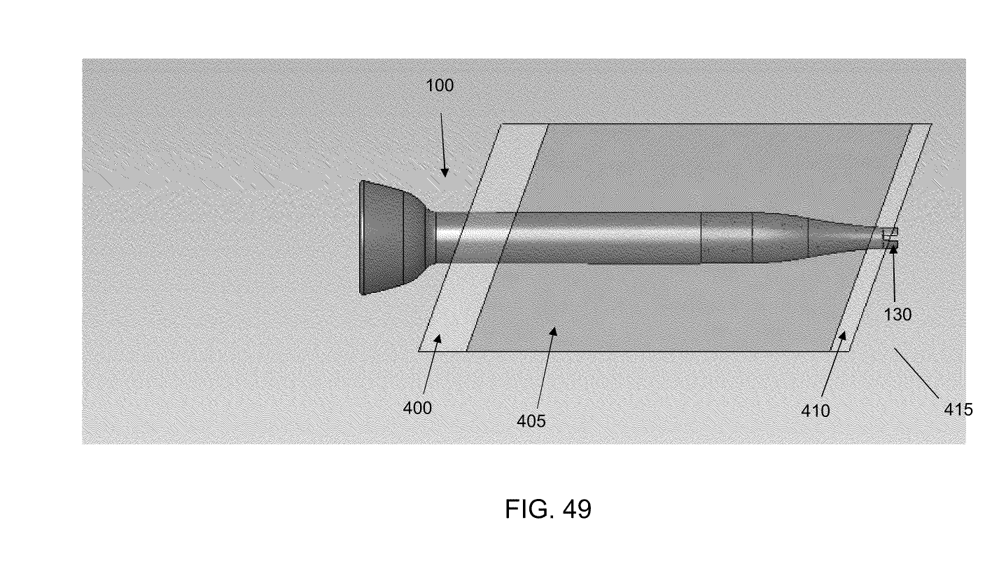

Removal of obturator 200 from access cannula 100 then leaves access cannula 100 in place so as to provide an access portal to the interior of the joint (FIG. 49). In this respect it will be appreciated that, as instruments are passed into and out of access cannula 100, membrane 145 permits the distal end of the access cannula to expand as needed so as to accommodate these instruments.

### Use of the Expandable Access Cannula for Other Joints, Etc.

It should be appreciated that laterally-expandable access cannula 5 and/or laterally-expandable access cannula system 95 may be used for accessing joints in addition to the hip joint, e.g., laterally-expandable access cannula 5 and/or laterally-expandable access cannula system 95 may be used to access the knee joint, the shoulder joint, etc. Furthermore, laterally-expandable access cannula 5 and/or laterally-expandable access cannula system 95 may also be used to access other interior bodily spaces, e.g., the abdomen, the bladder, regions around the spine, etc.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A laterally-expandable access cannula comprising:
an elongated body have a distal end, a proximal end and a lumen extending between the distal end and the proximal end, the distal end of the access cannula comprising a plurality of fingers tapering inwardly as they extend distally, and the elongated body having an internal thread extending along at least a portion of the length of the lumen; and
an inner sleeve disposed within the lumen of the elongated body, the inner sleeve having an external thread extending along at least a portion of its length, the external thread of the inner sleeve being in engagement with the internal thread of the elongated body, such that rotation of the inner sleeve causes the inner sleeve to move distally relative to the elongated body, whereby to cam open the plurality of fingers of the elongated body.

2. A laterally-expandable access cannula according to claim 1 wherein the inner sleeve includes at least one recess on its proximal end for engagement with a keyed driver.

3. A laterally-expandable access cannula according to claim 2 wherein the inner sleeve comprises a plurality of recesses on its proximal end for making a splined engagement with a keyed driver.

4. A laterally-expandable access cannula according to claim 2 wherein the keyed driver is separable from the access cannula.

5. A laterally-expandable access cannula according to claim 2 wherein the keyed driver is secured to the access cannula.

6. A laterally-expandable access cannula according to claim 1 further comprising a membrane extending about the plurality of fingers.

7. A method for accessing a joint, the method comprising:
providing a laterally-expandable access cannula comprising:
an elongated body have a distal end, a proximal end and a lumen extending between the distal end and the proximal end, the distal end of the access cannula comprising a plurality of fingers tapering inwardly as they extend distally, and the elongated body having an internal thread extending along at least a portion of the length of the lumen; and
an inner sleeve disposed within the lumen of the elongated body, the inner sleeve having an external thread extending along at least a portion of its length, the external thread of the inner sleeve being in engagement with the internal thread of the elongated body, such that rotation of the inner sleeve causes the inner sleeve to move distally relative to the elongated body, whereby to cam open the plurality of fingers of the elongated body;
inserting the laterally-expandable access cannula into tissue; and
advancing the inner sleeve within the lumen of the elongated body so as to cam open the fingers of the elongated body.

8. A method according to claim 7 wherein the inner sleeve includes at least one recess on its proximal end for engagement with a keyed driver.

9. A method according to claim 8 wherein the inner sleeve comprises a plurality of recesses on its proximal end for making a splined engagement with a keyed driver.

10. A method according to claim 8 wherein the keyed driver is separable from the access cannula.

11. A method according to claim 8 wherein the keyed driver is secured to the access cannula.

12. An access cannula comprising an elongated body having a distal end, a proximal end and a lumen extending between the distal end and the proximal end, the distal end of the access cannula comprising a plurality of flexible fingers tapering inwardly as they extend distally, with a membrane extending about the plurality of flexible fingers, so that the distal end of the access cannula normally has reduced inner and outer diameters relative to the remainder of the access cannula.

13. An access cannula according to claim 12 wherein the flexible fingers are spaced apart from one another so as to define slots therebetween.

14. An access cannula according to claim 12 wherein at least one seal is disposed across the lumen at the proximal end of the access cannula.

15. An obturator comprising an elongated body having a distal end, a proximal end and a lumen extending between the distal end and the proximal end, the distal end of the obturator comprising a conical structure followed by a plurality of spaced-apart fins.

16. An obturator according to claim 15 wherein the proximal end of the obturator comprises a handle having a lateral slot extending therethrough and communicating with the lumen.

17. An access cannula system comprising:
an access cannula comprising an elongated body having a distal end, a proximal end and a lumen extending between the distal end and the proximal end, the distal end of the access cannula comprising a plurality of flexible fingers tapering inwardly as they extend distally, with a membrane extending about the plurality of flexible fingers so that the distal end of the access cannula normally has reduced inner and outer diameters relative to the remainder of the access cannula; and
an obturator comprising an elongated body having a distal end, a proximal end and a lumen extending between the distal end and the proximal end, the distal end of the obturator comprising a conical structure followed by a plurality of spaced-apart fins.

18. The access cannula system of claim 17 wherein the obturator and the access cannula are sized so that (i) when the obturator is inserted within the lumen of the access cannula, the fins of the obturator are disposed between the flexible fingers of the access cannula; and (ii) when the obturator is rotated within the lumen of the access cannula, the fins of the obturator engage the flexible fingers of the access cannula and cam them laterally to an expanded-diameter configuration.

19. An access cannula system according to claim 17 wherein the flexible fingers of the access cannula are spaced apart from one another so as to define slots therebetween.

20. An access cannula system according to claim 17 wherein the distal ends of the flexible fingers of the access cannula are beveled inwardly.

21. An access cannula system according to claim 17 wherein at least one seal is disposed across the lumen at the proximal end of the access cannula.

22. An access cannula system according to claim 17 wherein the proximal end of the obturator comprises a handle having a lateral slot extending therethrough and communicating with the lumen.

23. An access cannula system according to claim 17 further comprising a guidewire.

24. An access cannula system according to claim 23 wherein the lumen of the obturator is sized to receive the guidewire therein.

25. An access cannula system according to claim 22 further comprising a guidewire, and further wherein the lateral slot of the obturator is sized to receive the guidewire therein.

26. A method for accessing a joint, comprising:
providing an access cannula system comprising:
an access cannula comprising an elongated body having a distal end, a proximal end and a lumen extending between the distal end and the proximal end, the distal end of the access cannula comprising a plurality of flexible fingers tapering inwardly as they extend distally, with a membrane extending over the plurality of flexible fingers so that the distal end of the access cannula normally has reduced inner and outer diameters relative to the remainder of the access cannula;
an obturator comprising an elongated body having a distal end, a proximal end and a lumen extending between the distal end and the proximal end, the distal end of the obturator comprising a conical structure followed by a plurality of spaced-apart fins; and
a guidewire;
passing the guidewire from outside the body, through intervening tissue and into the joint;
inserting the obturator within the lumen of the access cannula so that the fins of the obturator are disposed between the flexible fingers of the access cannula, with the membrane holding the distal end of the access cannula in a reduced-diameter configuration;
passing the access cannula/obturator assembly down the guidewire so that the access cannula/obturator assembly extends through the intervening tissue; and
rotating the obturator within the lumen of the access cannula so that the fins of the obturator engage and cam outboard the flexible fingers of the access cannula, with the membrane yielding so as to permit the distal end of the access cannula to enter an expanded-diameter configuration, whereby to dilate adjacent tissue.

27. A method according to claim 26 wherein the obturator and the access cannula are sized so that (i) when the obturator is inserted within the lumen of the access cannula so that the fins of the obturator are disposed between the flexible fingers of the access cannula, the membrane holds the distal end of the access cannula in a reduced-diameter configuration; and (ii) when the obturator is rotated within the lumen of the access cannula so that the fins of the obturator engage and cam them laterally to an expanded-diameter configuration.

28. A method according to claim 26 comprising the additional step wherein the guidewire is removed.

29. A method according to claim 26 comprising the additional step wherein the obturator is removed.

30. A method according to claim 26 comprising the additional step wherein the obturator is removed and then the guidewire is removed.

31. A method according to claim 26 comprising the additional step wherein an instrument is passed down the access cannula after the obturator is removed.
